(19) United States
(12) Patent Application Publication
(10) Pub. No.: US 2026/0224388 A1
(43) Pub. Date: Aug. 6, 2026

(54) EXOSKELETON SUPPORT DEVICE

(71) Applicant: Brian C Garcia, West Hurley, NY (US)

(72) Inventor: Brian C Garcia, West Hurley, NY (US)

(21) Appl. No.: 19/428,730

(22) Filed: Dec. 22, 2025

Related U.S. Application Data
(60) Provisional application No. 63/752,956, filed on Feb. 3, 2025.

(51) Int. Cl. A61F 5/37

(52) U.S. Cl. CPC A61F 5/373

(57) ABSTRACT

An exoskeleton support system is disclosed that provides ergonomic support for a user's arm, shoulder, and upper body during extended activities. The system includes a support plate having a ratcheted coupling portion that interlocks with an indexed pivot joint coupling to enable discrete angular adjustment. An arm assembly with a concave support member and a load-distribution support element is coupled to the pivot joint through an elongate slot member that provides linear adjustability. The load-distribution support element includes a connector stem and a padded upper surface configured to receive and support an elbow or forearm of the user. In certain embodiments, the connector stem employs a ball-and-socket joint to allow multi-directional adjustment and load balancing. The structure distributes weight through the frame to reduce muscular strain, promote proper posture, and enhance comfort for both seated and standing users.

### CROSS-REFERENCE TO RELATED APPLICATIONS

This application is a U.S. Non-Provisional Utility Patent Application entitled, “EXOSKELETON SUPPORT DEVICE” which claims priority to co-pending U.S. Provisional Patent Application No. 63/752,956, filed on Feb. 3, 2025 entitled, “Straight-N-UPR” the contents of which are hereby fully incorporated by reference.

### FIELD OF THE EMBODIMENTS

The present disclosure relates to personal health and ergonomic support systems. More particularly, the embodiments pertain to an exoskeletal assistive apparatus configured to promote proper posture and spinal alignment of a user by providing structural support to the back, neck, and upper limbs. The embodiments further relate to wearable support devices that reduce strain during use of handheld or entertainment electronic devices, thereby improving comfort, stability, and musculoskeletal wellness during prolonged seated or standing activities.

### BACKGROUND

Wearable support systems have been developed to address physical strain associated with poor posture and extended periods of sitting or standing. Conventional posture-correcting devices often consist of elastic braces or straps designed to align the shoulders and spine. These devices typically rely on passive tension and require manual adjustment to achieve the desired support. Although such braces may provide temporary relief, they frequently cause discomfort due to pressure points or restricted movement. In more advanced implementations, exoskeleton frameworks have been introduced to assist in physical rehabilitation, workplace ergonomics, and load-bearing applications. Industrial exoskeletons are commonly used to reduce lower-back stress in lifting tasks, while medical exoskeletons assist in gait training and mobility restoration. However, most commercial systems are bulky, powered by external actuators, and not intended for everyday or sedentary use.

Other known devices focus on improving arm and neck posture when using entertainment or electronic devices such as smartphones, tablets, or gaming controllers. These solutions generally employ adjustable stands or neck-mounted supports to reduce fatigue. Nonetheless, they lack integrated spinal alignment features and often fail to maintain ergonomic balance for the user's entire upper body. The current art therefore includes a range of posture-correcting braces, rehabilitative exoskeletons, and device-specific supports, each addressing limited aspects of ergonomic health. However, existing systems tend to prioritize either mobility assistance or localized limb support, and few provide comprehensive, lightweight, and user-friendly solutions suitable for daily postural correction and comfort.

### SUMMARY OF THE EMBODIMENTS

In some aspects, the techniques described herein relate to an exoskeleton support device, including: a support plate with a proximal end located opposite a distal end, the support plate including: an opening located at the distal end of the support plate; and a first ratcheted coupling portion located at the proximal end of the support plate; an indexed pivot joint coupling having a first end located opposite a second end, the indexed pivot coupling including: a second ratcheted coupling portion located at the first end of the indexed pivot joint coupling, wherein the second ratcheted coupling portion of the indexed pivot joint coupling is configured to interlock at one or more discrete angular positions with the first ratcheted coupling portion of the support plate; and one or more arms each having a concave support member, the one or more arms being connected to the second end of the indexed pivot joint coupling; an elongate slot member having a first end located opposite a second end, the first end of the elongate slot member being connected to the support plate; and a load-distribution support element connected to the second end of the elongate slot member.

In some aspects, the techniques described herein relate to an exoskeleton support device, wherein the first ratcheted coupling portion of the support plate having a plurality of radial teeth.

In some aspects, the techniques described herein relate to an exoskeleton support device, wherein the second ratcheted coupling portion of the indexed pivot joint coupling having a plurality of radial teeth.

In some aspects, the techniques described herein relate to an exoskeleton support device, wherein the second end of the indexed pivot joint coupling includes an opening configured to receive a threaded fastener.

In some aspects, the techniques described herein relate to an exoskeleton support device, further including: an opening located at the second end of the indexed pivot joint coupling configured to receive at least a portion of a threaded fastener for connecting at least one arm of the one or more arms to the indexed pivot joint coupling.

In some aspects, the techniques described herein relate to an exoskeleton support device, further including: a first arm of the one of more arms is disposed over a surface of a second arm and is configured to expand and retract to accommodate differing lengths.

In some aspects, the techniques described herein relate to an exoskeleton support device, wherein the elongate slot member is configured to provide linear adjustability between a pivot joint and a fixed mounting point.

In some aspects, the techniques described herein relate to an exoskeleton support device, wherein the load-distribution support element further includes a connector stem extending from a lower portion of the load-distribution support element and configured to couple the load-distribution support element to the second end of the elongate slot member to permit rotational or angular adjustment of the load-distribution support element relative to the elongate slot member.

In some aspects, the techniques described herein relate to an exoskeleton support device, wherein the load-distribution support element includes a concave upper surface configured to receive and cradle a hand of a user.

In some aspects, the techniques described herein relate to an exoskeleton support system, including: a frame configured to be worn by a user; a support plate having a proximal end located opposite a distal end, the support plate including a ratcheted coupling portion positioned at the proximal end; an indexed pivot joint coupling having a first end located opposite a second end, the first end of the indexed pivot joint coupling including a complementary ratcheted coupling portion configured to interlock with the ratcheted coupling portion of the support plate at one or more discrete angular positions; an arm assembly connected to the second end of the indexed pivot joint coupling, the arm assembly including a concave support member configured to support an arm region of the user; an elongate slot member having a first end located opposite a second end, the first end of the elongate slot member being connected to the support plate; and a load-distribution support element connected to the second end of the elongate slot member, the load-distribution support element including a padded upper surface configured to distribute load from the arm region of the user and a connector stem configured to couple the load-distribution support element to the elongate slot member.

In some aspects, the techniques described herein relate to an exoskeleton support system, wherein the padded upper surface of the load-distribution support element includes a resilient material selected from the group consisting of foam, gel, and elastomeric compounds.

In some aspects, the techniques described herein relate to an exoskeleton support system, wherein the connector stem includes a ball-and-socket joint configured to permit multi-directional angular adjustment of the load-distribution support element relative to the elongate slot member.

In some aspects, the techniques described herein relate to an exoskeleton support system, wherein the ball-and-socket joint further includes a locking mechanism configured to secure the load-distribution support element at a desired angular orientation.

In some aspects, the techniques described herein relate to an exoskeleton support system, wherein the elongate slot member includes graduated position markings configured to indicate relative adjustment positions of the load-distribution support element along a linear path.

In some aspects, the techniques described herein relate to an exoskeleton support system, wherein the concave support member of the arm assembly includes a replaceable ergonomic pad detachably coupled to an upper surface of the concave support member.

In some aspects, the techniques described herein relate to an exoskeleton support system, wherein the arm assembly further includes an outer tubular member and an inner telescoping member configured to adjust a length of the arm assembly.

In some aspects, the techniques described herein relate to an exoskeleton support system, wherein the inner telescoping member includes a detent locking pin configured to secure the arm assembly at one or more discrete extension positions.

In some aspects, the techniques described herein relate to an exoskeleton support system, wherein the support plate and the indexed pivot joint coupling are formed of lightweight composite or polymeric material configured to reduce overall system weight.

In some aspects, the techniques described herein relate to an exoskeleton support system, wherein the ratcheted coupling portion of the support plate and the complementary ratcheted coupling portion of the indexed pivot joint coupling are configured to lock at angular increments between about 5 degrees and about 15 degrees.

In some aspects, the techniques described herein relate to an exoskeleton support system, wherein the frame, the arm assembly, and the elongate slot member are cooperatively configured to transfer and distribute mechanical load from the load-distribution support element through the frame to a torso region of the user, thereby reducing localized strain on an elbow, forearm, or wrist of the user.

### BRIEF DESCRIPTION OF THE DRAWINGS

The present disclosure may be better understood, and its numerous features and advantages made apparent to those skilled in the art, by referencing the accompanying drawings. The use of the same reference symbols in different drawings indicates similar or identical items.

FIG. 1A illustrates a perspective rear view of an exoskeleton support device, according to some embodiments.

FIG. 1B illustrates a perspective side view of the exoskeleton support device of FIG. 1A, according to some embodiments.

FIG. 1C illustrates a side plan view of the exoskeleton support device of FIG. 1A, according to some embodiment.

FIG. 2 illustrates a perspective view of the support plate of the exoskeleton support device of FIG. 1A, according to some embodiments.

FIG. 3 illustrates a perspective view of the indexed pivot joint coupling of the exoskeleton support device of FIG. 1A, according to some embodiments.

FIG. 4 illustrates a perspective front view of one or more arms of the exoskeleton support device of FIG. 1A, according to some embodiments.

FIG. 5A illustrates an exploded view of a multi-arm configuration of the exoskeleton support device, according to some embodiments.

FIG. SB illustrates a front perspective view of the multi-arm configuration of the exoskeleton support device of FIG. 5A, according to some embodiments.

FIG. 6 illustrates a perspective view of an elongate slot member of the exoskeleton support device of FIG. 1A, according to some embodiments.

FIG. 7 illustrates a perspective view of a load-distribution support element of the exoskeleton support device of FIG. 1A, according to some embodiments.

FIG. 8 illustrates a perspective view of a spacer of the exoskeleton support device, according to some embodiments.

FIG. 9 illustrates a perspective view of an adjustable chest strap of the exoskeleton support device, according to some embodiments.

FIG. 10 illustrates an example embodiment of a user wearing the exoskeleton support device during operation, according to some embodiments.

FIG. 11 illustrates an example embodiment of a user wearing the exoskeleton support device during operation, according to some embodiments.

### DETAILED DESCRIPTION

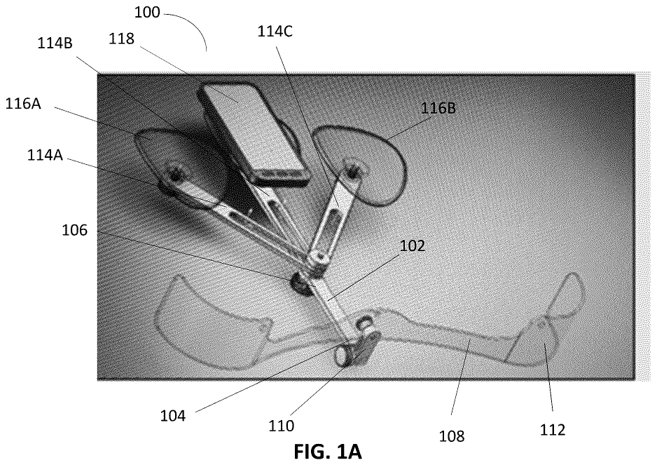

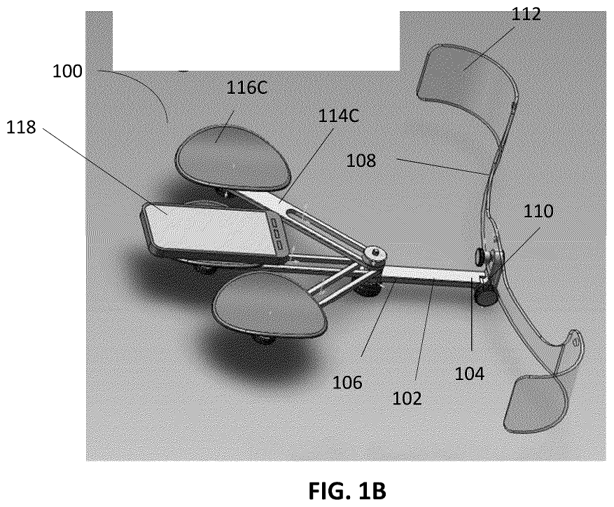

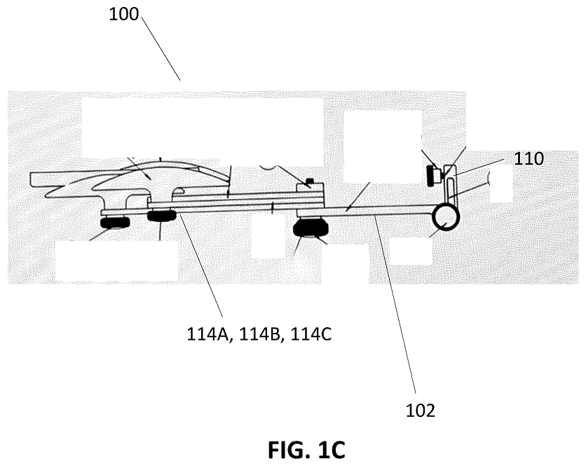

FIGS. 1-11 illustrate example systems and devices of an exoskeleton support device configured to provide ergonomic support and load distribution for a user's upper body. The embodiments shown depict a modular and adjustable wearable system designed to stabilize the user's arms, forearms, or hands during various daily or occupational activities. The exoskeleton support device may include a combination of structural components, such as a support plate, indexed pivot joints, elongate slot members, and load-distribution pads, configured to reduce muscle strain, promote proper posture, and enhance comfort during extended use. The figures collectively demonstrate multiple configurations, materials, and user-interactive arrangements suitable for both rehabilitative and assistive applications, showing how the device adapts to different user anatomies and task requirements.

A particular problem addressed by the disclosed system is “text neck,” a musculoskeletal condition associated with sustained forward flexion of the cervical spine that occurs when individuals habitually look downward at handheld electronic devices such as smartphones, tablets, or e-readers. This repetitive posture increases the effective load on the neck and upper back by altering the alignment of the head relative to the shoulders. At a forward inclination of about 15 degrees, the head exerts an effective force of about 12 kilograms (about 27 pounds) on the cervical spine, and at about 60 degrees, the load can exceed about 25 kilograms (about 60 pounds). Over time, this excessive strain may lead to microtrauma of cervical muscles, disc compression, and/or postural imbalance, which collectively contribute to symptoms such as chronic neck pain, shoulder tightness, upper back fatigue, and/or headache.

The exoskeleton support device 100 disclosed herein directly addresses these biomechanical stresses by elevating and stabilizing handheld electronic devices within the user's natural line of sight, thereby minimizing the need for forward head flexion. The system's adjustable slot members and pivot couplings allow the supported device to be positioned at an ergonomic viewing angle, typically within about 10 to about 15 degrees below eye level, reducing cervical spine flexion and promoting neutral spinal alignment. This posture aligns the head over the shoulders, allowing the spine to bear the head's weight efficiently rather than relying on continuous muscular tension.

Additionally, by mechanically supporting the user's forearms and hands, the device transfers load-bearing responsibilities from the smaller stabilizing muscles of the upper back and shoulders to the more robust torso structure. The load-distribution support elements and elongate slot members maintain the user's arms in a semi-flexed, neutral position, thereby minimizing static muscle activation in the trapezius, levator scapulae, and sternocleidomastoid muscles. The forearm support components may be adjusted within a vertical range of about 50 mm to about 150 mm and a horizontal range of about 80 mm to about 180 mm, allowing users of varying statures to maintain optimal ergonomic alignment. The arm angle may be positioned between about 60 and about 100 degrees relative to the upper torso, minimizing joint strain while providing natural motion flexibility.

As a result, the exoskeleton support device significantly reduces cumulative fatigue, mitigates the risk of repetitive strain injury, and supports long-term postural correction. In certain embodiments, users may experience a reduction of about 40 to about 60 percent in upper trapezius muscle activation when compared to unsupported conditions, as determined through electromyographic analysis. Through this combination of ergonomic positioning, load redistribution, and mechanical stabilization, the exoskeleton support device provides a comprehensive solution to the physical demands imposed by modern digital device usage, addressing both the causal mechanics and the symptomatic outcomes of text neck. The system therefore enhances comfort, reduces fatigue, and promotes long-term spinal health through user-adjustable, posture-corrective mechanical support.

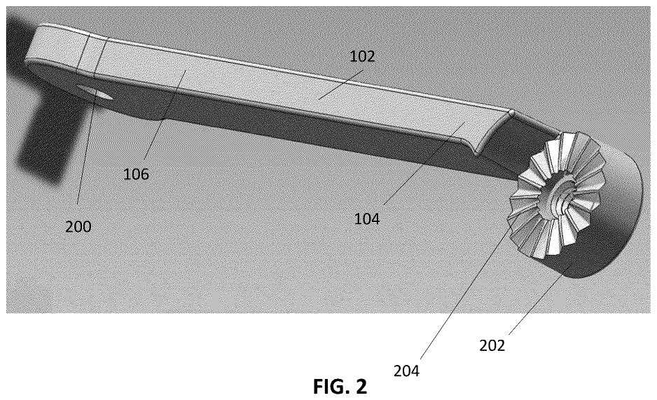

Referring to FIGS. 1A, 1B, and 1C, an exoskeleton support device 100 is shown for providing ergonomic support and load distribution for a user's arms, wrists, or electronic accessories. As best shown in FIG. 1A, the device 100 includes a support plate 102 having a proximal end 104 located opposite a distal end 106. The support plate 102 defines an opening 200 (FIG. 2) at the distal end 106 and a first ratcheted coupling portion 202 (FIG. 2) at the proximal end 104. The first ratcheted coupling portion 202 comprises a plurality of radial teeth 204 (FIG. 2) configured to engage complementary teeth on an indexed pivot joint coupling 110 to enable discrete angular positioning and locking between the two members.

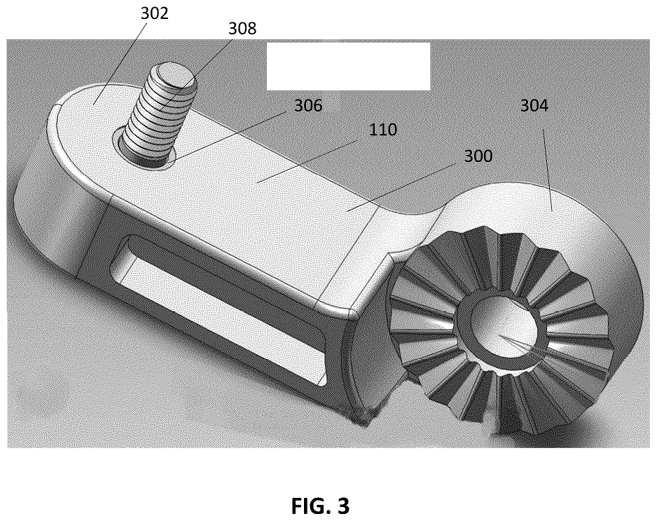

The indexed pivot joint coupling 110 includes a first end 300 (FIG. 3) opposite a second end 302 (FIG. 3). A second ratcheted coupling portion 304 (FIG. 3) located at the first end 300 interlocks with the first ratcheted coupling portion 202 of the support plate 102. This interlocking mechanism allows the angular orientation of the support plate 102 to be adjusted relative to the pivot coupling 110 in one or more discrete positions, maintaining stability during use. Connected to the second end 302 of the indexed pivot joint coupling 110 are one or more arms 108, each terminating in a concave support member 112. The concave support members 112 are contoured to cradle and support portions of the user's arms or elbows, distributing weight away from the shoulders and neck to improve posture and reduce fatigue. In some embodiments, the arms 108 may be telescopic or adjustable in length to accommodate users of varying stature.

Extending from the support plate 102 are multiple elongate slot members 114A-114C, each having a first end 600 and a second end 602. The first end 600 of each elongate slot member 114A-114C is coupled to the support plate 102, while the second end 602 connects to one or more load-distribution support elements 116A, 116B. The elongate slot members 114A-114C permit linear and angular adjustment of the load-distribution elements 116A, 116B relative to the support plate 102.

Each load-distribution support element 116A, 116B may comprise a mounting interface or pad configured to receive and support various objects. In certain embodiments, the load-distribution support elements 116A, 116B include a connector stem 118 configured to permit angular articulation and rotational positioning. The load-distribution support elements 116A, 116B may support one or more of a user's hands or wrists, or external items such as a book, tablet, phone, or electronic device, thereby enabling hands-free operation while maintaining balanced ergonomic posture. The device 100 thus provides an adjustable, modular, and lightweight exoskeleton-type framework that transfers mechanical load from the arms or supported objects through the structural elements 102, 110, 114A-114C and into the user's torso or frame interface. This configuration assists in maintaining proper spinal alignment, reducing localized muscle strain, and improving comfort during extended use in seated or standing positions.

The exoskeleton support device 100 provides multiple technical advantages over conventional arm rests, posture aids, and wearable support systems. The integration of the indexed pivot joint coupling 110 with interlocking ratcheted portions 202 (FIGS. 2) and 304 (FIG. 3) enables precise angular adjustment between connected structural members while maintaining rigid positional stability under load. This configuration allows the user to fine-tune ergonomic positioning and maintain posture alignment during varying activities such as reading, typing, or using an electronic device. The inclusion of elongate slot members 114A-114C introduces linear adjustability and multi-axis motion control, permitting the load-distribution support elements 116A and 116B to be repositioned along adjustable travel paths. This feature enhances load adaptability, allowing the device to accommodate different users, arm lengths, and activity types without requiring structural replacement or reassembly.

The concave support members 112 and load-distribution support elements 116A, 116B provide biomechanical weight transfer, distributing the gravitational load of the user's arms, wrists, or supported objects through the exoskeletal frame rather than through the user's muscles. As a result, the device reduces muscle fatigue in the neck, shoulder, and upper back regions and improves postural equilibrium during prolonged seated or standing use. The modular configuration of the support plate 102, elongate slot members 114A-114C, and pivot joint coupling 110 allows for interchangeable and scalable assembly, enabling the system to be adapted for consumer electronics, medical rehabilitation, or industrial applications. The ratcheted and slotted adjustment mechanisms further allow tool-free reconfiguration, improving user convenience and operational safety. Overall, the system offers a lightweight, durable, and dynamically adjustable mechanical structure that enhances ergonomic support, optimizes load distribution, and extends usability across multiple working and recreational environments.

FIG. 1B illustrates a perspective side view of an exoskeleton support device 100, according to one embodiment, showing a configuration in which each primary component is adjustable, rotatable, or slidably traversable to accommodate users of differing body sizes and ergonomic preferences. The exoskeleton support device 100 includes a support plate 102 having a proximal end 104 located opposite a distal end 106. The support plate 102 may be formed from aluminum alloy 6061-T6, titanium grade 2, carbon-fiber composite, or reinforced thermoplastic (e.g., polyamide 6/6 with 30-40% glass fiber content). The thickness of the support plate may range from 2 mm to 6 mm, more preferably from 3 mm to 5 mm, providing a high stiffness-to-weight ratio. In some embodiments, the plate is anodized or powder-coated for corrosion resistance and surface hardness. The plate may further include curved sidewalls or contoured relief sections to enhance load distribution while maintaining aesthetic form.

At the proximal end 104, the support plate 102 includes a first ratcheted coupling portion configured to engage an indexed pivot joint coupling 110. The pivot joint coupling 110 has a first end 300 with a second ratcheted coupling portion 304, which mates with the ratcheted teeth of the support plate 102. Each ratcheted coupling portion may include 18-40 radial teeth, preferably between 24-32 teeth, arranged circumferentially to allow discrete angular positioning in increments between 5° and 15°, more preferably about 10°. The coupling 110 may include a spring-loaded locking knob or torque-retention screw that selectively engages the ratchet interface. When tightened, the joint maintains rigid locking strength exceeding 25-50 N·m, preventing unintentional rotation during use.

The indexed pivot joint coupling 110 may further incorporate a dual-axis rotation mechanism, allowing both yaw and pitch adjustments through integrated bearings or nested hinge elements. This design enables the connected arm assemblies 108 to pivot laterally up to ±45°, rotate axially up to 90°, and tilt vertically within a range of 0°-60°, permitting natural human motion without mechanical interference. Coupled to the second end 302 of the pivot joint coupling 110 are one or more arms 108, each terminating in a concave support member 112. Each arm 108 may be formed as a tubular telescoping assembly, with an outer member having an inner diameter between 10 mm and 25 mm and an inner sliding member with a corresponding outer diameter sized for a clearance fit of 0.1-0.3 mm. The arms may extend over an adjustable length between 150 mm and 350 mm, more preferably between 200 mm and 300 mm, depending on user size and task orientation. The telescoping interface may include incremental detents, spring buttons, or threaded locking collars, allowing both fixed and continuous length adjustment.

Each arm 108 is further rotatably connected to the pivot joint coupling 110 through a clevis or trunnion joint that provides axial rotation of 180°-270°, more preferably about 225°, allowing the concave support members 112 to swing outward or inward relative to the user's body. This articulation enables the device to accommodate different reach angles, seated postures, or standing activities. The concave support members 112 are ergonomically contoured to cradle an elbow, wrist, or forearm, thereby reducing localized pressure points. Each support member 112 may include a rigid base layer formed from polycarbonate, aluminum, or magnesium alloy, overlaid with a resilient cushioning layer comprising viscoelastic polyurethane foam, gel-infused silicone, or thermoplastic elastomer (TPE). The upper surface may be covered by a breathable woven fabric or synthetic leather to improve comfort and moisture management. The curvature of each concave member 112 may have a radius of curvature between 60 mm and 120 mm, overlapping with the anatomical range of forearm diameters, thereby ensuring proper biomechanical load transfer.

Extending from the distal region 106 of the support plate 102 are elongate slot members 114A-114C, each having a first end 600 connected to the plate and a second end 602 connected to load-distribution support elements 116A-116C. Each elongate slot member may include a slotted channel defining a travel path length between 80 mm and 160 mm, preferably between 100 mm and 140 mm, providing adjustable reach. A fastener, guide pin, or carriage bolt positioned through the slot enables slidable traversal along the longitudinal axis, allowing the user to shift the position of the load-distribution element 116C forward, backward, or diagonally relative to the main frame. The slot members may be constructed from carbon-fiber laminate, aluminum extrusion, or machined titanium, and may further include graduated measurement markings or friction-based clamps for quick positional indexing.

Each load-distribution support element 116C includes a connector stem 118 configured to engage the second end 602 of the elongate slot member 114C. The connector stem 118 may be coupled via a ball-and-socket joint, allowing rotation about at least three orthogonal axes, roll, pitch, and yaw, with a total range of angular adjustment between 0° and 45° in each direction. The joint may be tensioned through an internal compression spring or nylon friction washer, providing smooth but controlled motion. The load-distribution support elements 116A-116C may serve as support pads or platforms for a variety of objects or body parts, including a mobile phone, tablet, notebook, book, or the user's wrists and hands. Each element may include a soft-touch rubberized upper surface formed from silicone rubber (Shore A hardness 20-40) to prevent slippage and absorb vibration. In alternative embodiments, the elements may include magnetic inserts, elastic bands, or snap-fit retaining lips to secure electronic devices.

In operation, the combination of rotational, slidable, and adjustable connections among the support plate 102, pivot joint 110, arms 108, slot members 114A-114C, and load-distribution elements 116A-116C enables the user to fine-tune orientation and spacing in three dimensions. The system is therefore capable of continuous adjustment, incremental locking, and multi-axis articulation, offering superior versatility compared to static or single-axis supports. When assembled, the exoskeleton support device 100 forms a load-bearing network that redistributes weight and reaction forces through the structural frame, reducing strain on the upper extremities. In exemplary operation, the user may rotate the arms 108 outward, extend them to a selected length, slide the load-distribution element 116C forward along the slot 114C, and tilt the support pad into a desired angle, all while maintaining a neutral wrist and elbow alignment. This arrangement allows a single configuration to adapt seamlessly to varying users, workstations, and devices, providing ergonomic comfort, posture correction, and sustained load management during daily use.

In certain embodiments, the exoskeleton support device 100 and its associated components may be fabricated using a combination of metallic, polymeric, and composite materials, selected to optimize stiffness, strength-to-weight ratio, manufacturability, and user comfort. The support plate 102 and indexed pivot joint coupling 110 may be formed from CNC-machined aluminum 6061-T6, CNC-milled titanium grade 2, or carbon-fiber-reinforced epoxy composite, with overlapping material thicknesses ranging from 2.0 mm to 6.0 mm, more preferably from 3.0 mm to 5.0 mm. These materials provide structural rigidity while maintaining total system mass below approximately 1.5 kilograms, depending on device configuration.

The arms 108 may be extruded or pultruded into tubular profiles from aluminum, carbon fiber composite, or glass-fiber-reinforced nylon (PA6-30GF). The inner telescoping segments may be machined or molded to achieve a clearance tolerance of about 0.05-0.3 mm for smooth extension and retraction. The telescoping interfaces may be locked using stainless steel detent pins, spring-loaded plungers, or threaded collet clamps. Surfaces may be anodized, powder-coated, or treated with PVD coatings to enhance wear and corrosion resistance. The elongate slot members 114A-114C may be manufactured by stamping, laser cutting, or waterjet machining depending on the selected base material. For composite variants, the slot members may be formed by vacuum-assisted resin transfer molding (VARTM) or autoclave-cured lay-up using carbon or glass fiber prepregs. Slot geometry may incorporate reinforced ribs or flanged sidewalls to resist bending during sliding adjustments. Fasteners that traverse the slots may comprise shoulder bolts, T-head guide pins, or nylon-lined bushings to ensure low-friction travel and vibration damping.

The ratcheted coupling portions 202 and 304 may be produced by precision CNC gear hobbing, injection molding, or additive manufacturing (SLS or MJF) to achieve consistent tooth spacing and surface finish. Tooth geometry may vary from 30° to 60° flank angles with a pitch depth between 1.5 mm and 3.5 mm, enabling secure engagement under repetitive motion. The ratchet components may be fabricated from acetal (POM), nylon 12, or glass-filled PPS, each offering high wear resistance and self-lubricating properties. The concave support members 112 and load-distribution support elements 116A-116C may be produced through injection molding, thermoforming, or hybrid over-molding techniques. Each support member may include a rigid base shell made of polycarbonate (PC) or ABS, over-molded with a soft-touch elastomeric layer such as thermoplastic polyurethane (TPU), silicone rubber, or TPE. The over-mold thickness may range between 1.0 mm and 3.0 mm, providing both impact absorption and tactile comfort. The inner padding may be die-cut from open-cell polyurethane foam or gel-filled polymer layers having hardness ratings from Shore A 20 to Shore A 40.

The connector stem 118 and ball-and-socket joints may be precision-machined from stainless steel, brass, or reinforced nylon and may include PTFE washers, polymer bushings, or bronze sintered bearings for smooth rotational motion. These joints may provide angular travel within overlapping ranges of 0°-45°, 0°-60°, or 0°-90° depending on configuration, and may be retained using compression springs, friction screws, or snap-fit retaining rings. Assembly of the exoskeleton support device 100 may be accomplished through threaded fasteners, press-fit bushings, or ultrasonic welding (for polymer interfaces). In some embodiments, adhesive bonding using epoxy resin, cyanoacrylate, or UV-curable acrylic adhesives may supplement mechanical fastening for improved vibration resistance. Final alignment and calibration may be performed using adjustment jigs to ensure smooth articulation and consistent torque retention across moving joints.

Surface finishing may include powder coating, anodizing, or soft-touch polymer coatings to improve aesthetic appearance and user comfort. In some versions, select components, such as the one or more load-distribution support elements 11A-116C, may incorporate embedded sensors or micro-switches fabricated using printed conductive ink or thin-film strain gauges for integration with monitoring systems or smart-device interfaces. Through this combination of precision manufacturing, interchangeable materials, and modular assembly techniques, the exoskeleton support device 100 achieves high structural integrity, adjustable articulation, and long-term durability suitable for ergonomic, industrial, and rehabilitative applications.

In operation, the exoskeleton support device 100 may be used by a seated or standing user to reduce muscle fatigue and maintain upright posture while performing tasks such as reading, typing, viewing electronic media, or manipulating handheld devices. The following example illustrates typical use and adjustment sequences that demonstrate the system's versatility and mechanical adaptability. Prior to use, the user positions the support plate 102 near the chest or torso region, aligning the proximal end 104 and indexed pivot joint coupling 110 at an ergonomic height approximately between 0.8 meters and 1.2 meters, depending on the user's stature. The first ratcheted coupling portion 202 on the support plate 102 engages the second ratcheted coupling portion 304 of the pivot joint 110, allowing the user to set the angular alignment. In typical adjustment, the coupling may be locked at one of multiple discrete angles between 5° and 45°, more preferably between 10° and 25°, which positions the arms 108 in forward-reaching alignment relative to the user's body.

Once the angular orientation is selected, the user may extend or retract the arms 108 to a desired length. Each arm includes a telescoping inner segment slidable within an outer housing, offering an effective travel range of 150 mm to 350 mm, and more preferably 200 mm to 300 mm. The extension can be set using a detent pin, friction collar, or twist-lock mechanism, ensuring that the arms maintain consistent support during movement. The user can then rotate the arms 108 about their connection to the pivot joint coupling 110 by up to 180°-270°, depending on comfort or activity type. At the terminal end of each arm, the concave support member 112 may be adjusted through its rotatable or tiltable interface, which may include a ball-and-socket or hinge-type connector. The support member 112 can tilt upward or downward between 0° and 60° to conform to an individual's forearm or elbow curvature. The surface of each support member may include soft, gel-based or elastomeric padding, providing localized pressure reduction and improved comfort during long-duration use.

The elongate slot members 114A-114C provide linear travel to adjust reach and spacing of load-distribution support elements 116A-116C. During setup, the user may slide each load-distribution element forward or backward along the slots to customize the alignment with the user's forearm, hand, or electronic device. The sliding adjustment typically allows travel between 80 mm and 160 mm, with incremental locking positions every 10 mm to 20 mm or continuous locking via a friction-based tightening knob. The slot-based traversal enables the device to accommodate a range of body types, arm lengths, and workstation configurations.

Each load-distribution support element 116C is connected via a connector stem 118, which allows the element to pivot about three orthogonal axes. The user can therefore rotate the load-distribution element 116C horizontally by up to ±45°, tilt vertically between 0° and 60°, and swivel laterally up to 90° relative to the elongate slot. This multi-axis articulation enables precise positioning for various activities—such as holding a tablet at reading height, supporting the forearms during keyboard entry, or resting the hands while gaming or sketching on a touchscreen surface. For instance, when the user employs the device to support a tablet or book, the load-distribution element 116C may be positioned at an upward tilt angle between 15° and 35° relative to the horizontal plane, with the arms 108 extended forward approximately 250 mm. This configuration aligns the user's forearms parallel to the ground and relieves static muscular tension from the deltoid and trapezius regions. In an alternate configuration for typing or workstation use, the user may lower the pivot joint 110 angle and retract the arms 108 slightly, positioning the concave support members 112 under the forearms and maintaining neutral wrist extension.

In another scenario, the exoskeleton support device 100 can assist users engaged in rehabilitation or assisted-mobility therapy. For example, when used by individuals with limited arm strength, the arms 108 may be adjusted to higher angular elevation (e.g., between 30° and 45°) to offload the majority of the arm's weight onto the frame. The ball-and-socket connectors 118 allow fine-tuned repositioning without excessive manual force, and friction-based dampers prevent sudden movements that could cause discomfort or instability. During extended use, the user may reconfigure the assembly without tools. The ratcheted pivot joint coupling 110 may be released by rotating a locking knob, enabling realignment of the support plate 102 within seconds. Similarly, the elongate slot members 114A-114C may be adjusted manually to reposition load-distribution elements for different device orientations. This tool-free modularity enhances the system's applicability across multiple contexts, including home, office, laboratory, or rehabilitation environments.

At the end of use, the system may be collapsed or folded by retracting the arms 108, unlocking the ratcheted coupling, and sliding the load-distribution elements 116C toward the support plate 102. The device's composite and aluminum components allow total weight to remain below 1.5-2.0 kilograms, making it portable and easy to store or transport. In summary, the exoskeleton support device 100 enables continuous ergonomic customization through a combination of rotatable, slidable, and telescoping components. Each linkage and pivot contributes to multi-axis articulation, allowing users to dynamically adjust support for their arms, wrists, or devices. The resulting system reduces fatigue, enhances postural alignment, and extends comfort across prolonged activity periods in occupational, recreational, or therapeutic applications.

While the embodiments described with reference to FIGS. 1A-1B illustrate exemplary configurations of the exoskeleton support device 100, numerous variations, substitutions, and equivalents may be implemented without departing from the inventive concept. In some embodiments, the exoskeleton support device 100 may include a miniaturized or compact configuration in which the support plate 102 and arms 108 are shortened for portable or desktop use. The reduced form factor may be adapted for mobile applications, such as a foldable travel version that fits within a laptop bag or backpack. The support plate 102 in these variants may be produced from additively manufactured polymer composites, such as PA12 nylon or carbon-fiber-reinforced PETG, fabricated by selective laser sintering (SLS) or fused deposition modeling (FDM) techniques.

In other embodiments, the indexed pivot joint coupling 110 may incorporate motorized or semi-active actuation to assist movement or maintain pre-set positions. A micro linear actuator or servo-driven hinge may be integrated within the joint housing to provide automated angular adjustment between 5° and 90°, responding to user input or preset ergonomic profiles. The drive system may be powered by a rechargeable lithium-ion battery with a capacity between 400 mAh and 1200 mAh, enclosed in the frame and controlled through low-voltage electronic circuitry. In yet another variation, the arms 108 may be equipped with gas-spring dampers or torsion-spring assist mechanisms to partially counterbalance the downward load of the user's arm. These configurations enable dynamic load balancing, maintaining a desired position with minimal muscular effort. The tension of such assist mechanisms may be user-adjustable, for example by a rotatable dial or threaded preload screw that modifies spring compression.

In some implementations, the concave support members 112 may feature interchangeable ergonomic pads of varying densities or profiles, enabling users to select configurations that match personal comfort or medical requirements. For clinical or rehabilitative use, the pads may be fabricated from medical-grade silicone elastomer or anti-microbial polyurethane foam with surface hardness ranging between Shore A 15 and Shore A 45. In addition, temperature-sensitive gel inserts may be incorporated to maintain thermal comfort during prolonged contact. The elongate slot members 114A-114C may also vary in number and configuration. In alternative embodiments, a single continuous slot rail or a dual-parallel linkage assembly may replace the independent slot members to improve structural symmetry and stability. These variations may employ linear bearings, roller tracks, or telescopic slides to permit smoother motion with reduced friction. The travel length of such mechanisms may extend from 50 mm to 200 mm, overlapping the range of the standard embodiment, and may include adjustable end stops or spring detents for controlled traversal limits. The load-distribution support elements 116A-116C may be adapted for multifunctional use. In some embodiments, the elements may include integrated mounting interfaces, such as magnetic plates, Velcro hook-and-loop surfaces, or retractable clips, allowing secure attachment of external devices such as smartphones, tablets, books, or instrumentation tools. The connector stem 118 may further include rotational torque sensors or strain gauges configured to detect user-applied forces and transmit feedback to a connected computing device.

In an advanced embodiment, the system may integrate sensor-based feedback control for posture correction. Embedded inertial measurement units (IMUs) or gyroscopic tilt sensors within the support plate 102 and pivot joint coupling 110 may monitor the user's position and generate feedback through a connected mobile application. The sensors may detect deviations exceeding 5°-10° from an optimal posture angle and prompt adjustment cues via haptic vibration or visual alerts. Alternative designs may substitute the mechanical ratchet interface between the support plate 102 and pivot joint coupling 110 with magnetically indexed locking mechanisms or toothless friction clutches, allowing continuous angular adjustment rather than discrete steps. Friction torque thresholds may be tuned between 0.5 N·m and 2.5 N·m, providing sufficient resistance to maintain static orientation while allowing deliberate repositioning by the user.

In some embodiments, the exoskeleton support device 100 may be configured for dual arm operation, wherein two mirror-image assemblies are symmetrically mounted to a central body harness or torso plate. The harness may be constructed from lightweight nylon webbing, Kevlar-reinforced fabric, or 3D-printed polymer mesh. This dual configuration allows balanced bilateral support, transferring forces evenly through both sides of the user's upper body. Additionally, the system may be scaled for industrial or occupational applications. Larger embodiments may employ reinforced steel pivot joints, extended slot members exceeding 250 mm, and high-load-rated support pads designed to support tools or instrumentation weighing between 0.5 kg and 3.0 kg. Such embodiments can assist in manufacturing, inspection, or laboratory work where sustained arm elevation is required.

Conversely, a miniature variant of the exoskeleton support device 100 may be adapted for wearable consumer electronics, such as head-mounted displays or lightweight sensor rigs, where only partial arm or wrist support is necessary. These smaller versions may employ micro hinge joints and shortened slot members made of polyether ether ketone (PEEK) or reinforced ABS, maintaining adjustability in a reduced footprint. Across all embodiments, the structural framework of the exoskeleton support device 100 remains modular and reconfigurable, with components that may be rotated, slid, extended, or replaced without specialized tools. This adjustability allows the system to accommodate multiple use environments, user anthropometries, and functional objectives while preserving the underlying ergonomic and mechanical principles of the invention.

The exoskeleton support device 100 offers numerous functional and industrial advantages that extend across ergonomic, medical, and occupational domains. By integrating ratcheted coupling, telescoping arm assemblies, elongate slot members, and multi-axis pivot joints, the device provides a highly adjustable and mechanically stable framework that reduces muscular strain while promoting neutral posture. Unlike traditional armrests or fixed support brackets, the disclosed system enables continuous, tool-free customization in angular, axial, and linear dimensions, allowing users to adapt positioning dynamically throughout prolonged use. From a technical standpoint, the combination of indexed angular locking and linear traversability ensures repeatable precision without loss of rigidity, even after extended operation cycles. The mechanical interfaces distribute load uniformly through the support plate and frame, thereby reducing concentrated stress points and extending product longevity. In clinical and rehabilitative applications, the device aids individuals recovering from shoulder or elbow injuries by offloading limb weight and maintaining consistent joint alignment.

Industrial applicability spans a broad range of sectors. In office and educational environments, the system functions as a portable ergonomic workstation accessory, improving comfort for typing, reading, or tablet interaction. In healthcare settings, the same adjustable structure serves as a rehabilitative exoskeletal aid facilitating gradual strength training and motion stabilization. In manufacturing or inspection contexts, larger embodiments provide assistive support for repetitive or overhead tasks, mitigating fatigue and occupational strain injuries. The modular nature of the design enables cost-effective fabrication and scalable assembly using standard processes such as CNC machining, extrusion, injection molding, and additive manufacturing. Components can be disassembled and replaced individually, minimizing maintenance cost and environmental waste. Furthermore, the adaptability of materials, ranging from lightweight composites for consumer versions to high-strength alloys for industrial models, ensures broad manufacturability across multiple production scales. Accordingly, the disclosed exoskeleton support device 100 represents an ergonomically advanced, materially efficient, and industrially versatile system capable of improving user comfort, extending endurance, and enhancing productivity in applications requiring sustained upper-body support.

Referring to FIG. 2, the support plate 102 of the exoskeleton support device 100 is shown in greater structural detail. The support plate 102 extends between a proximal end 104 and a distal end 106, defining a longitudinal axis along which operational loads are transmitted. The opening 200 formed at the distal end 106 is configured to receive a fastener, bushing, or connecting pin, enabling attachment to an elongate slot member or another frame component. At the proximal end 104, the first ratcheted coupling portion 202 forms part of the plate's pivot interface. The first ratcheted coupling portion 202 comprises a series of radial teeth 204 arranged circumferentially about the rotational axis of the plate. The radial teeth 204 are configured to interlock with complementary radial teeth of a corresponding coupling on the indexed pivot joint coupling (110, FIGS. 1A-1B). The engagement of these teeth allows for incremental angular adjustment and secure positional locking between adjacent components.

Each radial tooth 204 may have a pitch depth ranging from about 1.0 mm to 4.0 mm, preferably between about 1.5 mm and 3.5 mm, and more preferably between about 2.0 mm and 3.0 mm, depending on the torque retention and angular resolution required for a given configuration. The included flank angle of each tooth may range from about 25° to 70°, preferably between about 30° and 60°, and more preferably between about 35° and 50°, providing a balanced relationship between locking strength and disengagement smoothness. The tooth width measured at the pitch circle may be between about 1.0 mm and 2.5 mm, with a tooth height-to-spacing ratio between about 0.4:1 and 0.8:1, resulting in consistent surface contact and efficient load transfer across the coupling interface.

The geometry of the radial teeth 204 allows discrete angular positioning at increments between approximately 3° and 20°, preferably between about 5° and 15°, enabling precise ergonomic adjustment and repeatable alignment. In smaller or fine-adjustment embodiments, the ratcheting interface may provide incremental angles as low as 2° to 4°, achieved through higher tooth counts of approximately 36 to 72 teeth distributed circumferentially. In heavy-duty or industrial embodiments, the interface may provide coarser indexing between about 10° and 20°, with fewer but deeper teeth configured to withstand greater mechanical load. The radial teeth 204 may be precision-machined using CNC milling, wire electrical discharge machining (EDM), or gear-hobbing, achieving a surface roughness below Ra 0.8 μm when formed from metallic materials such as aluminum 6061-T6, stainless steel 17-4PH, or titanium grade 2. For polymeric or composite configurations, the teeth may be produced through injection molding, compression molding, or additive manufacturing processes such as selective laser sintering (SLS) or multi-jet fusion (MJF) using nylon 12, carbon-fiber-reinforced polyamide, or polyether ether ketone (PEEK).

In certain embodiments, the radial teeth 204 may be insert-molded or over-molded onto a metallic support plate 102, forming a hybrid coupling interface that combines structural rigidity with vibration damping. The polymeric insert may serve as a compliant tooth layer that absorbs impact and reduces noise during adjustment. Post-processing techniques such as hard anodizing, nitriding, or PTFE surface impregnation may be applied to improve wear resistance and reduce friction during repeated engagement. Dimensional tolerance between adjacent teeth may be maintained within ±0.02 mm to ±0.05 mm, preferably within ±0.03 mm, ensuring uniform interlocking and minimal backlash between the mating ratcheted portions. This precision allows the coupling interface to maintain repeatable angular stability through more than 50,000 engagement cycles, providing long-term mechanical reliability and consistent user performance.

In certain embodiments, the first ratcheted coupling portion 202 and the radial teeth 204 operate in conjunction with a locking and torque-retention mechanism configured to maintain the desired angular orientation once adjustment is achieved. This mechanism may include one or more compression elements, friction interfaces, or spring-biased detent assemblies that generate a clamping force sufficient to prevent unintentional rotation under operational loading. A representative embodiment employs a compression disc and threaded fastener assembly disposed along the central axis of the pivot interface. A fastener or bolt, typically fabricated from stainless-steel A2-70 or grade 8.8 alloy steel, may extend through a bushing or shoulder washer to apply axial load to the engaged ratchet surfaces. Tightening torque for this fastener may range from about 1.0 N·m to 4.5 N·m, preferably about 2.5 N·m to 3.5 N·m, producing a normal clamping force between approximately 300 N and 800 N at the tooth interface. This preload ensures that the engaged radial teeth 204 remain locked even when subject to dynamic torque loads up to about 25 N·m to 50 N·m during user movement or device repositioning.

In alternative configurations, a spring-loaded detent pin may replace the compression screw. The detent pin may include a stainless-steel plunger biased by a coil spring having a spring constant between about 2 N/mm and 6 N/mm, permitting manual retraction for angular repositioning. Upon release, the detent automatically re-engages one of several index holes or grooves positioned circumferentially around the pivot interface, providing tactile feedback and audible confirmation of secure engagement. The detent mechanism allows rapid repositioning without the need for tools while maintaining repeatable angular accuracy within ±2°. In another embodiment, a friction-damped clutch assembly may supplement or replace the ratchet locking feature. This clutch may consist of PTFE-lined friction washers, bronze sintered discs, or fiber-reinforced composite rings interposed between the mating ratchet faces. The friction coefficient may range between 0.18 and 0.35, generating a resistive moment that holds orientation under static conditions but permits deliberate repositioning when a threshold torque is exceeded.

Such a configuration provides smooth, continuous angular adjustment rather than discrete step indexing, suitable for applications requiring quiet or fine motion control. In hybrid embodiments, the ratcheted and friction-lock mechanisms may be combined, allowing the radial teeth 204 to provide primary mechanical engagement and the friction clutch to serve as a secondary damping interface that mitigates vibration and shock. Both locking types may employ self-lubricating bushings or low-friction polymer inserts (for example, acetal [POM] or polyimide [KAPTON]) to minimize wear and maintain consistent torque response across repeated adjustment cycles. The locking and torque-retention components may be enclosed within a protective housing integrated into the proximal end 104 of the support plate 102 or the corresponding body of the indexed pivot joint coupling 110. The housing may be sealed with O-rings or polymer gaskets to prevent ingress of dust and moisture, thereby extending service life in industrial or rehabilitative environments. This combination of mechanical engagement through the radial teeth 204 and controlled torque retention through compression, detent, or frictional mechanisms enables precise, durable, and user-adjustable angular stability that characterizes the exoskeleton support device 100.

The support plate 102 may be fabricated from aluminum alloy 6061-T6, titanium, or carbon-fiber-reinforced polymer composite, depending on weight and stiffness requirements. In alternate embodiments, the plate 102 may comprise glass-filled nylon (PA6-30GF) or polyether ether ketone (PEEK) for lightweight and high-impact resistance. The plate thickness may range from 2.0 mm to 6.0 mm, overlapping between 3.0 mm and 5.0 mm for optimal stiffness-to-weight balance. In some embodiments, the radial teeth 204 are integrally formed with the support plate 102 through machining or molding. In other embodiments, the teeth may be part of a separate metallic or polymeric insert press-fitted or bonded to the proximal end 104, allowing for component replacement or multi-material construction.

During assembly, the first ratcheted coupling portion 202 of the support plate 102 engages a corresponding ratcheted coupling on the indexed pivot joint coupling, allowing rotation about the pivot interface until a desired angular position is reached. Once aligned, the joint may be secured using a threaded fastener, compression screw, or spring-loaded locking mechanism, maintaining rigid stability under load while permitting reconfiguration when desired. This adjustable ratcheted coupling interface, defined by the radial teeth 204, provides precise and repeatable angular control, contributing to the overall ergonomic adjustability and mechanical stability of the exoskeleton support device 100.

Referring to FIG. 3, the indexed pivot joint coupling 110 serves as the primary mechanical interface between the support plate 102 and one or more arms 108 of the exoskeleton support device 100. It provides angular adjustability, torque retention, and structural continuity throughout the system. The indexed pivot joint coupling 110 includes a first end 300 located opposite a second end 302. The first end 300 comprises a second ratcheted coupling portion 304, which is configured to interlock with the first ratcheted coupling portion 202 and corresponding radial teeth 204 of the support plate 102, as previously illustrated in FIG. 2. The second ratcheted coupling portion 304 defines a circumferential array of radial teeth configured for precision engagement with the corresponding teeth of the first ratcheted coupling portion 202. Each radial tooth may have a pitch depth between about 1.0 mm and 4.5 mm, preferably between about 1.5 mm and 3.5 mm, and more preferably between about 2.0 mm and 3.0 mm, depending on material stiffness, torque requirement, and intended load capacity. The included flank angle of each tooth may range from about 20° to 75°, preferably between about 30° and 60°, and more preferably between about 35° and 50°, selected to balance ease of engagement with shear strength along the contact flanks. The root-to-tip radius may be between about 0.25 mm and 0.75 mm, with the tooth width at the pitch circle measuring between about 1.0 mm and 2.8 mm to ensure consistent bearing area under compression.

The number of teeth forming the circumferential array may vary according to the overall diameter of the coupling interface. Typical embodiments include between about 18 and 72 teeth, more commonly between about 24 and 48 teeth, with 32- or 36-tooth configurations offering a practical balance between mechanical strength and angular resolution. These values enable discrete angular indexing in selectable increments between approximately 2° and 20°, preferably between about 4° and 12°, and most preferably about 5° to 10° per adjustment step. Fine-adjustment versions, intended for ergonomic or lightweight consumer models, may employ higher tooth counts of 48 to 72 teeth to achieve micro-indexing increments as small as 2° to 4°, while industrial or heavy-duty variants may employ coarser spacing of 10° to 18° for increased torque retention.

The outer diameter of the ratcheted coupling portion 304 may range from about 20 mm to 60 mm, preferably between about 25 mm and 45 mm, with the tooth engagement depth comprising between about 20% and 40% of the total diameter. Axial thickness of the toothed section may range from about 3 mm to 10 mm, preferably between about 4 mm and 8 mm, allowing adequate interfacial surface area for load transmission while maintaining low device weight. The resulting coupling is capable of sustaining torsional loads between approximately 10 N·m and 60 N·m, depending on material selection, surface finish, and locking-fastener preload.

The tooth geometry may be optimized through finite-element analysis (FEA) to reduce local stress concentrations and ensure smooth load distribution during angular adjustment. The radial teeth 304 may be formed by CNC milling, gear-hobbing, wire EDM, or injection-mold tooling, achieving dimensional tolerances within ±0.02 mm to ±0.05 mm across the active engagement surface. Surface roughness may be controlled to Ra≤0.8 μm for metallic materials and Ra≤1.2 μm for polymeric materials, ensuring repeatable, low-friction engagement over more than 50,000 adjustment cycles. The interlocking geometry between the ratcheted portions ensures precise engagement and high torsional load resistance while permitting selective repositioning.

In some embodiments, the second ratcheted coupling portion 304 may undergo various surface treatments, coatings, or composite layering processes to enhance wear resistance, reduce friction, and improve long-term torque retention performance. When fabricated from metallic materials such as aluminum 6061-T6, stainless steel 17-4PH, or titanium grade 2, the ratcheted surface may be hard anodized to a thickness between about 25 μm and 75 μm, preferably between about 30 μm and 50 μm, resulting in surface hardness levels between approximately 350 HV and 500 HV. In some embodiments, plasma nitriding or gas carburizing may be used to induce case-hardening depths between about 0.1 mm and 0.4 mm, thereby increasing wear life in applications with repeated angular repositioning or high applied torque.

When the coupling portion 304 is produced from polymer or composite materials, such as glass-fiber-reinforced nylon, carbon-fiber-reinforced PEEK, or polyoxymethylene (POM), surface performance may be enhanced through the addition of PTFE fillers, graphite micro-particles, or molybdenum disulfide (MoS₂) dispersed uniformly within the bulk resin. These additives reduce the coefficient of friction to values between about 0.15 and 0.35, allowing smooth sliding engagement and minimizing galling at the interface with the first ratcheted coupling portion 202. The wear rate under dry contact conditions may thus remain below 10⁻⁶ mm³/N·m, ensuring stable engagement torque even after extended service.

In hybrid metal-polymer embodiments, the radial teeth may be formed as a two-layer or multi-layer composite structure, comprising a rigid metallic substrate overlaid with a thin polymeric or elastomeric contact layer. The polymeric overlay, typically between about 0.3 mm and 1.0 mm thick, may be injection over-molded or bonded to the metallic substrate using epoxy adhesive, thermal fusion, or co-curing processes. This layered configuration combines the mechanical strength and heat dissipation properties of metal with the low-friction, noise-damping, and self-lubricating behavior of the polymer.

Additional protective coatings may include diamond-like carbon (DLC) or titanium nitride (TiN) films deposited to thicknesses between about 1 μm and 5 μm using physical vapor deposition (PVD). Such coatings can achieve surface hardness values up to 1500 HV to 2000 HV, with friction coefficients as low as 0.1, improving torque retention repeatability and preventing micro-welding of the tooth flanks during high-load cycling. For polymeric or additive-manufactured couplings, the surface may be laser-sintered or micro-polished to remove stair-step texture and to reduce asperity contact. In certain embodiments, a fluoropolymer impregnation or silicone-based dry lubricant may be applied to the tooth faces at a thickness of 5 μm to 15 μm to provide long-lasting lubrication without contamination of surrounding components.

Testing has shown that combinations of these surface treatments and material pairings can maintain a torque-holding efficiency of 90% or greater after 50,000 to 100,000 engagement cycles, depending on the operational environment. The treated ratcheted coupling portion 304 therefore provides a stable, low-maintenance mechanical interface that resists wear, corrosion, and dimensional deformation while preserving precise angular indexing throughout the service life of the exoskeleton support device 100. A through-opening 306 extends along a longitudinal axis through the body of the pivot joint coupling 110 and is configured to receive a fastener 308, such as a machine screw, shoulder bolt, or detent-based locking shaft. In some embodiments, the fastener 308 provides the axial preload that presses the engaged ratchet surfaces together. When the fastener is tightened to a torque of about 2 N·m to 4 N·m, the resulting axial compression force may range from approximately 300 N to 700 N, maintaining the coupling in a fixed position during operation. In alternative configurations, the through-opening 306 may house a spring-loaded detent pin or friction clutch insert, providing adjustable resistance to rotation and enabling tool-free repositioning of the coupling relative to the support plate.

The body of the pivot joint coupling 110 may be fabricated from CNC-machined aluminum 6061-T6, stainless steel 17-4PH, or carbon-fiber-reinforced polymer composite, depending on the target weight and strength requirements. Metallic versions may include hard-anodized surfaces or PTFE-coated friction faces to reduce wear during repeated adjustment cycles. Polymeric variants may be injection-molded from glass-filled nylon 6/6 or polyether ether ketone (PEEK), with typical wall thicknesses between 2.0 mm and 4.5 mm to maintain stiffness while minimizing mass. At the second end 302, the coupling 110 may include an integral boss, clevis mount, or pivot sleeve configured to connect to an arm 108 or other articulating component of the system. The pivot interface may allow rotational travel of up to 180°, enabling folding, extension, or lateral repositioning of the attached arm. The coupling geometry may further include filleted transitions and reinforced sidewalls to distribute stress evenly under bending and torsional loads.

The second ratcheted coupling portion 304 and the first ratcheted coupling portion 202 collectively define an indexed interface that can be adjusted repeatedly without loss of precision. The coupling may incorporate self-lubricating bushings, nylon washers, or bronze thrust bearings between the engaged surfaces to achieve consistent torque performance and smooth engagement. In some embodiments, an elastomeric friction pad may be positioned behind the ratcheted interface to provide damping and noise reduction during adjustment. In operation, the indexed pivot joint coupling 110 allows the user to rotate the arms 108 or connected components relative to the support plate 102 through controlled, discrete angular increments. Once the desired alignment is achieved, the fastener 308 or detent assembly locks the interface, maintaining stable orientation under static and dynamic loading. This mechanism provides a durable, repeatable, and adjustable connection that forms the functional core of the exoskeleton's load-distribution architecture.

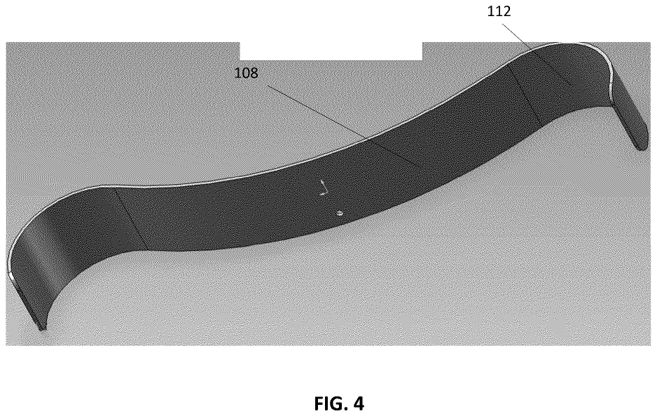

Referring to FIG. 4, the arm 108 of the exoskeleton support device 100 is shown in a front view, illustrating its curvature, width profile, and integration with a concave support member 112. The arm 108 provides a key structural component configured to extend between the indexed pivot joint coupling 110 (FIG. 3) and the distal support interface that contacts the user's forearm or elbow. Each arm 108 may be formed as a single continuous member or as an assembly of telescoping or hinged segments. In some embodiments, the arm 108 defines a gradual concave curvature having a radius of curvature between about 100 mm and 400 mm, preferably between about 150 mm and 250 mm, corresponding approximately to the ergonomic contour of a human forearm. The curvature allows the support surface to follow the natural posture of the user's arm, thereby reducing localized pressure and promoting even weight distribution.

The cross-sectional width of the arm 108 may range from about 20 mm to 60 mm, preferably between about 30 mm and 45 mm, and the thickness may be between about 2.0 mm and 6.0 mm, depending on the structural material used. The arm may include a central reinforcement rib or internal cavity to reduce mass while maintaining flexural rigidity. In adjustable configurations, the arm 108 may include nested telescoping sections having an extension range between 100 mm and 250 mm, allowing the user to fine-tune the reach of the concave support member 112 relative to the main frame.

The arm 108 may be fabricated from a variety of lightweight and durable materials. Metallic embodiments may include CNC-milled aluminum 6061-T6, titanium grade 5, or stainless steel 304, optionally surface treated with anodizing, bead blasting, or powder coating for corrosion resistance and aesthetic uniformity. Non-metallic embodiments may utilize carbon-fiber-reinforced epoxy, glass-fiber-reinforced nylon, or high-modulus thermoplastic polymers such as PEEK or polycarbonate, each providing strength-to-weight ratios suitable for wearable or portable configurations. The concave support member 112, located at the distal portion of the arm 108, defines a curved cradle that supports the user's elbow, forearm, or wrist. The concave surface may have a radius of curvature between about 50 mm and 120 mm, preferably between about 60 mm and 90 mm, corresponding to the typical anatomical contour of the forearm region. The support member may include a multi-layer ergonomic interface, such as a rigid base plate made of polycarbonate or aluminum overlaid with viscoelastic or memory-foam padding and a soft outer covering made from breathable fabric, silicone, or thermoplastic elastomer (TPE).

In some embodiments, the concave support member 112 may be rotatably or pivotally connected to the distal end of the arm 108, allowing angular adjustment through an arc of between about 20° and 90°, more preferably between about 30° and 60°, to align with the user's natural arm orientation. The rotational interface may incorporate a ball-and-socket connector, hinged pin joint, or detent-based pivot. The connector may further include a locking knob or friction clutch that secures the support member at the desired angle, with a torque retention capability between about 1.5 N·m and 4.0 N·m. The concave support member 112 may also include an embedded damping insert, such as a silicone gel pad or polyurethane foam core, having a compressive deflection between about 10% and 25% under a standard 50 N load, allowing the surface to conform to the user's arm while reducing vibration and static pressure. The resulting configuration provides comfort during extended periods of use, such as reading, typing, or operating handheld devices. The combination of the arm 108 and concave support member 112 therefore enables the exoskeleton support device 100 to provide adjustable, ergonomic, and stable upper-limb support, reducing fatigue, enhancing posture, and distributing loads efficiently through the system's structural framework.

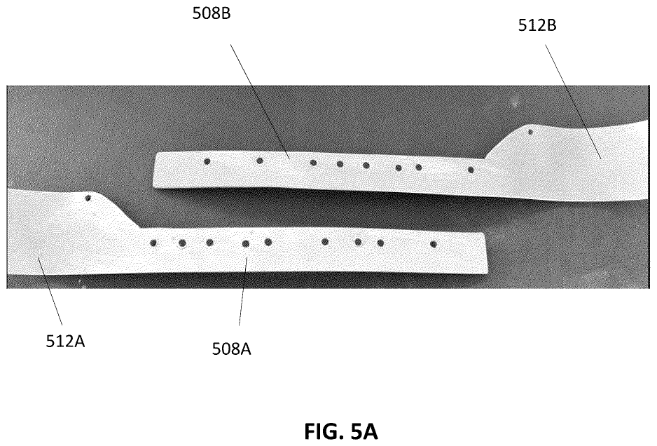

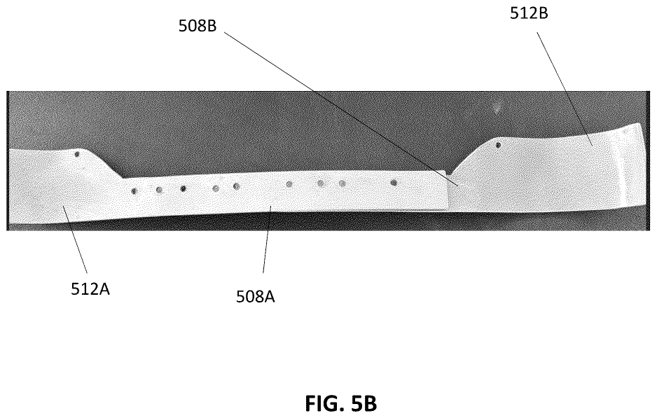

Referring to FIG. 5A, the multi-arm configuration of the exoskeleton support device 100 is shown, comprising a first arm 508A and a second arm 508B, each independently connected to the support plate 102 through respective coupling interfaces. The two arms 508A and 508B are arranged in mirror symmetry relative to the central longitudinal axis of the support plate 102, enabling the device to provide balanced, bilateral support for both of the user's forearms. Each arm 508A, 508B comprises an elongate frame defining multiple adjustment apertures distributed along its length. These apertures are configured to receive fasteners, locking pins, or sliding pegs that enable the arms to be extended, retracted, or repositioned to suit user-specific anthropometric measurements. The number of adjustment apertures may range from 5 to 12, preferably between 6 and 10, with center-to-center spacing between about 15 mm and 30 mm, allowing for fine incremental length control. The total effective extension range of each arm may be between 100 mm and 250 mm, depending on device size and application type.

The proximal portions 512A and 512B of the arms are configured to connect to corresponding pivot interfaces or coupling brackets of the support plate 102 via threaded fasteners, clevis pins, or rotary bushings. The coupling interface allows each arm to articulate through an angular range between about 90° and 180°, more preferably between about 120° and 160°, permitting forward, lateral, and downward positioning. This adjustability enables ergonomic alignment for different postures, such as seated, standing, or semi-reclined operation. The arms 508A and 508B may be fabricated from lightweight high-strength materials, such as aluminum alloy 6061-T6, titanium, carbon-fiber-reinforced polymer (CFRP), or glass-fiber-reinforced nylon (PA6-30GF). The arm thickness may range from 2.0 mm to 5.0 mm, with a width between about 25 mm and 50 mm, providing the required bending stiffness while maintaining low weight. The arms may also include filleted transitions at the proximal and distal ends to minimize stress concentration and fatigue under cyclic loading.

In some embodiments, the arms may be formed as two-piece telescoping assemblies, with the inner segment sliding within an outer sleeve. The telescoping interface may provide continuous adjustability with a travel distance between about 100 mm and 200 mm, secured by a thumb-screw clamp, spring-loaded pin, or detent-based friction collar. When locked, the telescoping section can resist axial pullout forces of 150 N to 400 N, ensuring stable operation under normal ergonomic loads. The apertures formed along the arms 508A, 508B may have diameters between about 3 mm and 8 mm, preferably between 4 mm and 6 mm, and may be countersunk or chamfered to accommodate low-profile fasteners. In some versions, the apertures may be internally threaded or fitted with bushings or inserts made of bronze or PTFE-lined stainless steel to reduce friction and prevent galling during repeated adjustments.

The distal ends of the arms may be configured to mount the concave support members 112 (see FIG. 4), which support the user's forearms or wrists. These attachments may include rotational joints allowing angular adjustment between about 15° and 45°, enabling the user to fine-tune contact angle and orientation. The proximal ends 512A, 512B may further include curved attachment brackets or hinge joints, allowing rotational freedom while maintaining lateral rigidity. During assembly, the first and second arms 508A, 508B are secured to the support plate 102 via corresponding fasteners that engage the pivot joint coupling 110. The symmetrical arrangement of the two arms allows each side to operate independently yet maintain mechanical balance across the central frame. This configuration enables one arm to support a mobile device, while the opposite arm provides ergonomic support for the user's forearm or elbow, or both arms may be configured to operate synchronously for dual-limb support.

In operation, each arm may be extended or retracted independently to accommodate the user's reach and activity type. For instance, during seated workstation use, the arms 508A and 508B may be adjusted to equal lengths, providing uniform horizontal support. In contrast, during standing or angled applications, one arm may be shortened relative to the other to achieve a tilted ergonomic position that stabilizes the forearms at differing heights. The bilateral arm assembly of FIG. 5A thereby enables customizable, balanced, and dynamically adjustable upper-limb support, promoting proper spinal alignment and reducing muscular fatigue during prolonged activities. The combination of symmetrical geometry, incremental length control, and articulating couplings enhances both the functionality and adaptability of the exoskeleton support device 100 across a wide range of ergonomic applications.

Referring to FIG. 5B, the first arm 508A and second arm 508B of the exoskeleton support device 100 are shown in a front perspective view illustrating their telescoping and overlapping configuration. In this embodiment, the first arm 508A is disposed over the surface of the second arm 508B such that the two arms form a slidable coupling assembly allowing for variable extension and retraction along a common longitudinal axis. This arrangement enables the overall arm length to be adjusted smoothly to fit users of differing sizes or to adapt to specific activity requirements, such as typing, reading, or tool operation.

The arms 508A and 508B each include a series of aligned adjustment apertures, designated 512A and 512B, distributed along their length. These apertures are positioned to receive retention fasteners, locking pins, or quick-release detents that secure the two arms in one of several discrete length positions. The number of apertures may range from 6 to 12, preferably between 8 and 10, and may be spaced at intervals between about 15 mm and 25 mm, thereby providing multiple incremental positions for precise length adjustment. When the apertures of the two arms are aligned and locked, the arms maintain structural rigidity comparable to a single continuous member.

The first arm 508A may have an internal guide slot or channel formed along its inner surface, while the second arm 508B includes a corresponding tongue or guide rail that fits within the slot, maintaining linear alignment during telescoping movement. The guide interface may have a clearance tolerance between about 0.1 mm and 0.3 mm to permit smooth motion while minimizing lateral play. The total range of extension between the retracted and fully extended positions may be between 100 mm and 250 mm, preferably between 125 mm and 200 mm, providing sufficient adjustment for varying forearm lengths and ergonomic configurations.

Each arm 508A and 508B may be fabricated from aluminum alloy 6061-T6, carbon-fiber-reinforced epoxy laminate, or glass-fiber-reinforced nylon (PA6-30GF). Metallic embodiments may include bead-blasted or anodized surfaces to improve corrosion resistance and reduce friction between the sliding components. Polymeric or composite versions may include integrated PTFE fillers or silicone-based surface coatings to achieve low-friction self-lubrication during extension and retraction. The proximal end 512A of the first arm 508A and the proximal end 512B of the second arm 508B are configured to couple with respective pivot or hinge interfaces located near the indexed pivot joint coupling 110 (FIG. 3). This connection allows each arm pair to articulate jointly or independently through a rotational range between about 90° and 180°, depending on user adjustment. The distal ends of the arms (not shown in FIG. 5B) may connect to concave support members 112 (FIG. 4), enabling angular adjustment between about 15° and 60° relative to the horizontal plane.

When the first arm 508A is retracted over the second arm 508B, the assembly achieves a compact configuration suitable for storage or low-reach tasks. When extended, the arms provide increased span for taller users or applications requiring a broader range of motion. The telescoping mechanism can support tensile loads between about 100 N and 300 N and torsional loads between about 10 N·m and 25 N·m, depending on the locking mechanism and material composition. In some embodiments, the telescoping assembly may include a spring-loaded friction pad or compression insert between the two arms to provide damping during motion and to hold the arms in intermediate positions. The interface may employ a nylon washer, elastomeric pad, or silicone strip to maintain quiet operation and resist vibration. The dual-arm telescoping design of FIG. 5B therefore allows the exoskeleton support device 100 to be length-adjustable, modular, and easily reconfigurable, providing personalized ergonomic alignment for users of varying anthropometric dimensions. The combination of incremental aperture locking, guided telescoping motion, and independent arm articulation ensures precise adjustment and stable support, improving comfort and reducing muscular fatigue during extended use.

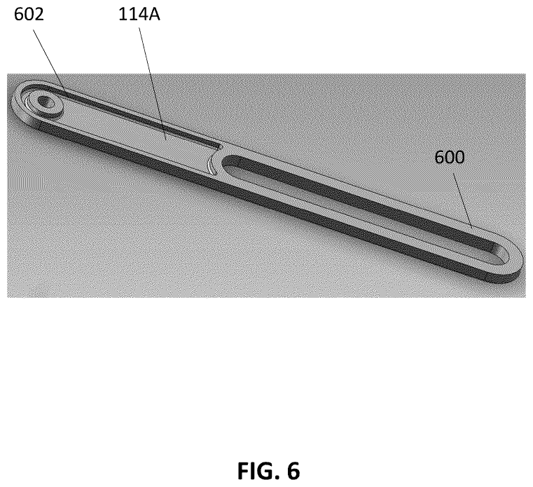

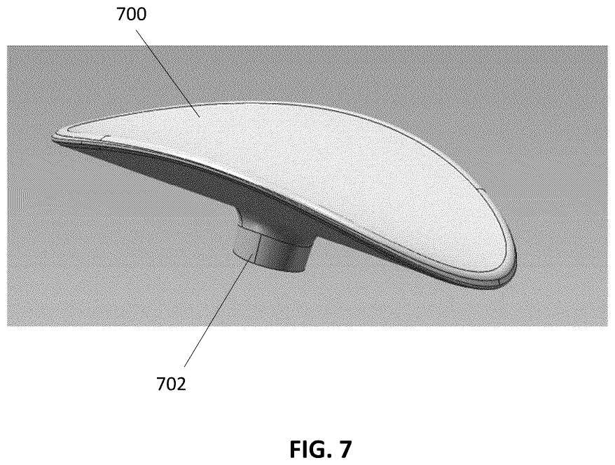

Referring to FIG. 6, the elongate slot member 114A of the exoskeleton support device 100 is shown in detail. The elongate slot member 114A functions as a linear adjustment arm enabling variable positioning between the structural frame and the load-distribution elements of the system. The elongate slot member extends between a first end 600 and a second end 602, defining a longitudinal axis along which a connector element may slide to adjust the relative distance between the connected components. The first end 600 may be configured with an opening or bushing adapted to receive a fastener, pivot pin, or bearing that connects the elongate slot member to the support plate 102 (FIGS. 1A-1B). The second end 602 may include a circular aperture or terminal bearing seat configured to couple to a connector stem 118 or to the load-distribution support element 116C (FIG. 7). The intermediate section of the elongate slot member 114A defines a slotted channel configured to receive a guide bolt, sliding fastener, or carriage pin, enabling linear translation of one end relative to the other.

The slot length may range from about 80 mm to 180 mm, preferably between about 100 mm and 140 mm, allowing fine adjustment for different user positions or support configurations. The slot width may range between about 6 mm and 14 mm, preferably between about 8 mm and 10 mm, providing clearance for a fastener or pin head while maintaining lateral stability during motion. The thickness of the elongate slot member may be between about 2.5 mm and 6.0 mm, depending on the selected material and load requirements. The slot geometry may include rounded terminal ends to minimize stress concentration and extend fatigue life. In some embodiments, the slot may incorporate graduated position markings, indicia, or laser-etched reference scales to assist users in returning to preferred positions. The sliding interface may employ low-friction washers, linear bushings, or polymer inserts fabricated from materials such as PTFE, UHMW polyethylene, or nylon 66 to ensure smooth adjustment with minimal wear.

The elongate slot member 114A may be fabricated from a variety of structural materials depending on application and weight targets. Exemplary materials include CNC-machined aluminum 6061-T6, titanium grade 2, or stainless steel 304 for high-load industrial embodiments, and carbon-fiber-reinforced polymer (CFRP) or glass-fiber-reinforced nylon (PA6-30GF) for lightweight ergonomic embodiments. Composite or polymer versions may be produced through injection molding, resin transfer molding, or additive manufacturing (SLS or MJF) techniques. Metallic versions may undergo anodizing, powder coating, or PTFE surface treatment to enhance corrosion resistance and reduce frictional drag between contacting parts.

In certain configurations, the first end 600 of the elongate slot member 114A may be fixed to the support plate 102 via a pivot coupling that allows limited angular motion, typically between 10° and 30°, to accommodate compound movement between the arm assemblies and the load-distribution support elements. The second end 602 may be connected via a rotational or ball joint coupling, allowing the load-distribution element to articulate about multiple axes. The sliding fastener assembly may include a threaded bolt with a friction washer or spring-loaded clamp knob, allowing the user to lock or unlock the sliding position manually. When tightened, the clamping force may range between about 150 N and 400 N, preventing unintended movement under operational loads. Under typical ergonomic applications, the slot member can withstand bending moments of 5 N·m to 20 N·m and tensile loads between about 200 N and 600 N without deformation.

In operation, the elongate slot member 114A allows the connected load-distribution support element 116C to slide forward or backward relative to the support plate 102, providing continuous adjustability in reach and position. This adjustability enables the user to fine-tune the spatial alignment of the support pads relative to their forearms, wrists, or mounted device, achieving optimal ergonomic comfort and load balance. Once the desired position is reached, the clamping fastener or detent mechanism can be engaged to secure the assembly in place. The design of the elongate slot member 114A therefore provides a durable, lightweight, and user-adjustable linkage within the exoskeleton support device 100. By combining linear traversal, rotational coupling, and secure clamping mechanisms, the slot member enhances the modularity and ergonomic adaptability of the overall system.

Referring to FIG. 7, the load-distribution support element 700 is shown in detail. The support element 700 provides the user-interface surface for the exoskeleton support device 100 and is designed to distribute contact forces evenly across the user's forearm or hand while maintaining adjustability relative to the elongate slot member 114A (FIG. 6). The load-distribution support element 700 may have a planform shape that is generally oval, elliptical, or teardrop-like, with a major axis length between about 80 mm and 160 mm, preferably between about 100 mm and 130 mm, and a minor axis width between about 50 mm and 100 mm, preferably between about 65 mm and 85 mm. The overall thickness of the element may range from about 10 mm to 35 mm, preferably between about 15 mm and 25 mm, depending on the materials and internal layering. The upper surface of the element is concavely contoured with a radius of curvature between about 40 mm and 120 mm, preferably between about 60 mm and 90 mm, providing a shallow cradle for ergonomic contact.

The upper surface may be constructed as a multi-layer assembly with a rigid structural base made of polycarbonate (PC), aluminum 6061-T6, or carbon-fiber-reinforced polymer (CFRP), an intermediate cushioning layer made of viscoelastic polyurethane foam, gel-infused silicone, or ethylene-vinyl acetate (EVA) composite, typically between about 3 mm and 8 mm thick, and an outer comfort layer of thermoplastic elastomer (TPE), neoprene, or microfiber fabric, providing tactile comfort, breathability, and slip resistance. The connector stem 702 extends downward from the underside of the load-distribution support element 700 and serves as the mounting interface with the elongate slot member 114A. The connector stem may be cylindrical, conical, or ball-shaped and may include a male coupling feature configured to engage a female socket or rotational joint at the second end 602 of the slot member (FIG. 6). The diameter of the connector stem 702 may range from about 8 mm to 20 mm, preferably between about 10 mm and 15 mm, and the axial height may range from about 15 mm to 40 mm.

The connection between the connector stem 702 and the elongate slot member 114A may provide rotational movement about a vertical axis and angular tilt adjustment about one or more transverse axes. The rotational range may extend between ±45° and ±90°, while the tilt range may extend between about 10° and 60°. The joint may include PTFE washers, nylon bushings, or spherical bearings to reduce friction and maintain smooth articulation. In some embodiments, a friction clutch or detent mechanism may be incorporated into the coupling to allow the user to lock the load-distribution support element 700 at a desired orientation. The locking torque may range from about 1.0 N·m to 3.5 N·m, ensuring stable positioning under typical ergonomic loads.

The underside of the load-distribution support element 700 may include reinforcement ribs or a honeycomb structure to improve stiffness while minimizing weight. The element may be manufactured through injection molding, compression molding, or additive manufacturing techniques. In composite versions, layers may be co-cured to form a monolithic shell. Metallic variants may be CNC-machined and then over-molded with the cushioning and comfort layers. In some embodiments, the load-distribution support element 700 may include sensor integration, such as force-sensitive resistors (FSRs), capacitive touch sensors, or temperature sensors, embedded beneath the upper surface. These sensors may detect pressure distribution or contact duration, allowing feedback to be transmitted to a control system or mobile device for ergonomic assessment or user interaction logging.

During operation, the load-distribution support element 700 functions as the final point of contact between the user and the exoskeleton support device 100. The concave form evenly disperses loads over a larger surface area, reducing localized pressure on the user's forearm or wrist. The adjustable connector stem 702 allows the element to follow the natural movement of the arm while maintaining support, thereby enhancing comfort, reducing muscular fatigue, and promoting proper posture during extended use. The combination of ergonomic contouring, adjustable articulation, and multi-material construction enables the load-distribution support element 700 to serve as both a structural and comfort component within the exoskeleton support device 100, ensuring stability, adaptability, and long-term durability under repeated dynamic use.

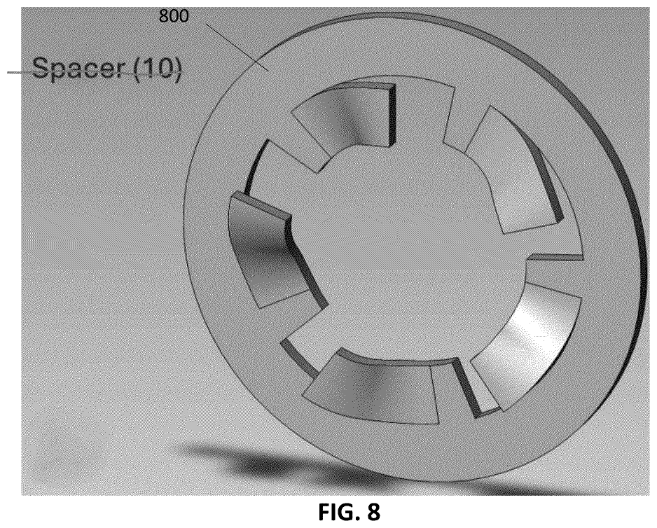

Referring to FIG. 8, the spacer 800 is shown as a structural interface component for the exoskeleton support device 100. The spacer 800 is generally circular in shape and defines a ring-like body having a smooth outer cylindrical surface and a smooth inner cylindrical bore. The central opening is honed or machined to a precise inner diameter configured to receive a knob stud, bolt, or pivot pin, allowing it to serve as a bushing or separation element between adjacent mechanical components. The outer diameter of the spacer 800 may range from about 15 mm to 40 mm, preferably between about 20 mm and 30 mm, while the inner diameter may range between about 6 mm and 16 mm, preferably between about 8 mm and 12 mm. The axial thickness of the spacer may range between about 1.5 mm and 6.0 mm, depending on the spacing requirement between the connected arm flex adjuster forearms. The inner bore is honed to achieve a surface finish smoother than Ra 0.8 μm, providing minimal friction and ensuring concentric alignment during rotational motion.

The spacer 800 may be fabricated from engineering-grade polymeric materials such as nylon (PA6 or PA66), polyoxymethylene (POM), acetal copolymer (Delrin®), or ultra-high-molecular-weight polyethylene (UHMWPE). These materials offer low-friction characteristics, dimensional stability, and high wear resistance during repeated movement cycles. For lightweight applications, the spacer may alternatively be formed from reinforced thermoplastic composites, including glass-filled polypropylene or carbon-fiber-filled polycarbonate, providing improved stiffness while maintaining low mass.

In alternative embodiments, the spacer 800 may be fabricated from metallic materials, including aluminum alloy 6061-T6, brass, or stainless steel 304, for applications requiring higher load-bearing capacity. In such configurations, a surface treatment such as anodizing, PTFE coating, or electropolishing may be applied to reduce friction and prevent corrosion. The central through-opening of the spacer 800 is configured to receive a knob stud, bolt, or pivot pin that passes through the aligned holes of the arm flex adjuster forearms. The spacer ensures consistent separation between the arms, maintaining parallel alignment and preventing over-compression when the knob or bolt is tightened. When assembled, the spacer 800 may be positioned concentrically about the central pivot axis, allowing the arms to rotate smoothly relative to one another without binding or surface wear.

In the illustrated embodiment, the inner bore of the spacer may include rounded edge fillets or chamfers at the top and bottom faces to assist in self-centering during assembly. The outer cylindrical surface may include a slight interference fit or press-fit tolerance of about 0.02 mm to 0.05 mm relative to the housing bore or the arm recess, depending on the design requirements. The smooth finish on both inner and outer surfaces enables low-friction rotation and minimizes noise during movement. When multiple arm flex adjuster forearms are used in a stacked configuration, such as for dual-arm or multi-axis articulation, the spacer 800 is inserted between them to maintain proper alignment and spacing. The spacer prevents surface contact and wear between adjacent arms and allows uniform tightening of the knob stud across the assembly. In typical use, one or more spacers 800 may be positioned along the pivot joint axis, depending on the number of forearm components in the configuration.

In operation, the spacer 800 functions as a bushing-like intermediary that allows the connected arm assemblies to flex, pivot, or rotate smoothly while maintaining stable mechanical spacing. The use of a polymeric or self-lubricating material further reduces friction, eliminates the need for external lubrication, and ensures quiet and efficient motion of the exoskeleton's arm system. The spacer 800 thus provides a durable, lightweight, and low-maintenance component essential for proper alignment, reduced friction, and consistent torque performance in the multi-arm configuration of the exoskeleton support device 100.

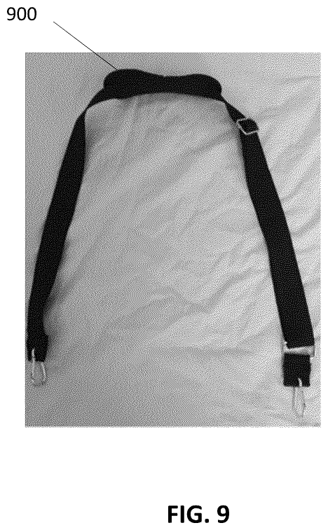

Referring to FIG. 9, the adjustable chest strap 900 is shown as a wearable support component designed to secure and stabilize the exoskeleton support device 100 against the user's upper torso. The chest strap 900 extends horizontally across the chest region and wraps rearward around the left and right upper arms or biceps, providing balanced anchoring of the device while maintaining user comfort and freedom of movement. The chest strap 900 may be formed from a flexible, high-strength, polymeric or composite material, such as nylon webbing, polyester fiber, thermoplastic polyurethane (TPU) laminate, or elastic woven polyethylene terephthalate (PET). The strap width may range from about 25 mm to 60 mm, preferably between about 35 mm and 45 mm, and the thickness may range from about 1.5 mm to 4.0 mm, depending on material selection and load-bearing requirements. The strap may exhibit a tensile strength between about 800 N and 2500 N, ensuring reliable performance under extended use.

A plurality of adjustment holes or slots are formed along the length of the chest strap 900 to allow for incremental length adjustment. The number of adjustment holes may range from 5 to 15, preferably between 8 and 12, with spacing between about 20 mm and 30 mm. These holes may receive snap fasteners, button pins, or buckle prongs to allow for quick resizing to accommodate users of varying chest circumferences. In some embodiments, the strap may incorporate a ratcheting buckle system or hook-and-loop interface for continuous adjustability. At the center of the chest strap 900, a pivot connector (clicker) or mounting hub is positioned to interface with other elements of the exoskeleton support device 100. The clicker may include a rotatable joint, snap-lock mechanism, or quick-release coupling, allowing for modular connection of adjustable components such as the support plate 102, indexed pivot joint coupling 110, or arm assemblies 108. The clicker may be made from injection-molded acetal, glass-filled nylon, or anodized aluminum, with a diameter between about 20 mm and 40 mm. The mechanism may include an internal detent or spring latch to hold the attached component securely while still permitting disengagement when required.

The terminal ends of the chest strap 900 may include metal or polymer D-rings, quick-connect buckles, or swivel hooks configured for adjustable attachment around the user's arms or to the exoskeleton's frame. These connectors may be constructed from stainless steel, aluminum alloy, or reinforced plastic, and are designed to resist tensile forces between 200 N and 800 N during motion. An optional neck strap may be removably connected to the chest strap 900 via a pair of attachment loops or snap connectors positioned near the upper shoulder regions. The neck strap may extend behind the user's neck to provide vertical stabilization, maintaining alignment of the exoskeleton frame against the user's torso. The neck strap may be fabricated from the same material as the chest strap and may include elastic or padded inserts to improve comfort and reduce pressure on the trapezius region.

The inner surface of the chest strap 900 may include a soft lining or non-slip interface layer to improve grip against clothing or skin. Suitable materials include neoprene, silicone-coated fabric, or microcellular foam with a surface friction coefficient greater than 0.6, reducing slippage during movement. The strap may also incorporate ventilation perforations or mesh panels to enhance breathability during prolonged use. During operation, the chest strap 900 functions as an anchoring system that distributes the mechanical load generated by the exoskeleton support device 100 evenly across the user's chest and shoulders. When properly adjusted, the strap allows the device to remain centered on the torso, preventing unwanted shifting while still permitting upper-body rotation and arm extension. The pivot connector allows dynamic coupling with other components, enabling a full range of ergonomic and motion-assisted configurations. The adjustable chest strap 900 thus provides a lightweight, comfortable, and highly adaptable mounting interface for the exoskeleton support device 100. Its combination of adjustability, flexibility, and modular connectivity allows users to achieve stable, ergonomic fitment while performing various seated, standing, or motion-intensive tasks.

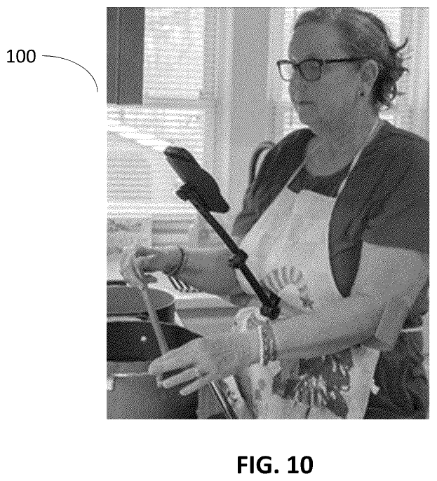

Referring to FIG. 10, the exoskeleton support device 100 is shown worn by a user during active use. In this example, the device supports an electronic device mounted to the elongate slot member 114A, enabling the user to view and interact with the device without manual holding. The illustrated embodiment demonstrates a real-world application in which the system provides ergonomic forearm and wrist support during prolonged tasks, such as cooking, reading, or device operation, while maintaining upright posture and minimizing upper-body fatigue. The elongate slot member 114A, as previously described in FIG. 6, provides linear adjustability between a proximal end attached to the support plate 102 and a distal end 602 that interfaces with a load-distribution support element or mounting assembly for the electronic device. The slot channel of member 114A allows translational movement along its longitudinal axis, permitting the user to position the mounted device at varying distances and angles. The adjustment range may span between 80 mm and 180 mm, preferably between 100 mm and 140 mm, to accommodate different user heights and working postures.

The electronic device mounted to the distal end of the elongate slot member 114A may be secured by a universal clamp, threaded adapter, or magnetic interface. The coupling may allow multi-axis adjustment, including rotation of approximately 180° about a vertical axis and tilt adjustment of 0° to 90° about a horizontal axis. This flexibility allows the user to orient the device for optimal viewing during varied activities, such as referencing recipes, video conferencing, or controlling connected systems. The exoskeleton support device 100 transmits mechanical load through the support plate 102, indexed pivot joint coupling 110, and elongate slot member 114A, reducing static muscular effort in the deltoid and trapezius muscles. The system may include a pair of concave arm supports 112 (FIG. 4) or load-distribution elements 700 (FIG. 7) configured to cradle the user's forearms and maintain neutral alignment during operation. This structure enables extended use without discomfort or fatigue, allowing both hands to remain free for fine-motor activities.

In the illustrated embodiment, the chest strap 900 (FIG. 9) that typically anchors the device around the user's torso is not shown for visual clarity. When present, the chest strap provides lateral stability and distributes the load across the upper torso. Without the strap shown, the figure emphasizes the relationship between the elongate slot member 114A, the pivot couplings, and the electronic device in use. The hidden structural connection may include a pivot clicker or central connector that couples the upper frame of the device to the torso harness, maintaining overall stability during movement. The materials of construction for the exoskeleton support device 100 may include lightweight aluminum alloys, carbon-fiber-reinforced polymers, or glass-filled thermoplastics to ensure mechanical strength while keeping the total system weight below 1.5 kilograms. Joints and interfaces, including the ratcheted couplings (202, 304) and rotational pivots (110, 602), may incorporate nylon bushings, PTFE washers, or bronze bearings to reduce friction and improve durability under repeated use.

In practical operation, as shown in FIG. 10, the exoskeleton support device 100 allows the user to perform hands-free interaction with an electronic device at eye or chest level while maintaining neutral joint alignment. The elongate slot member 114A and associated articulation points may be adjusted to raise, lower, or laterally reposition the supported device. This capability improves accessibility and reduces strain associated with prolonged handheld use, particularly in occupational, rehabilitative, or home environments. By enabling adjustable, posture-friendly support for the upper limbs and mounted devices, the exoskeleton support device 100 enhances user comfort and efficiency during both stationary and dynamic activities. The embodiment shown in FIG. 10 thus demonstrates the system's capacity to maintain ergonomic stability and adaptability across multiple real-world applications, while minimizing muscular fatigue and promoting upright posture.

The exoskeleton support device 100 disclosed herein provides a range of technical and ergonomic advantages over conventional posture aids, wearable arm supports, and handheld device mounts. The device integrates multi-axis mechanical adjustability, lightweight structural materials, and ergonomically contoured interfaces to deliver adaptive, posture-corrective support across a variety of activities and user profiles. One primary technical advantage is the multi-degree-of-freedom adjustability achieved through the combination of the support plate 102, indexed pivot joint coupling 110, and elongate slot members 114A-114C. This configuration enables controlled rotational, linear, and angular adjustment throughout the device, allowing the user to customize the orientation of the supported load relative to their torso. Angular indexing of between 2° and 20°, combined with linear adjustability of between 80 mm and 180 mm, provides fine-grained positioning suitable for users of differing anthropometric dimensions and postural preferences. The ratcheted coupling system (202, 304) featuring radial teeth 204 offers precise and repeatable angular positioning with high torsional stability. This indexed interface ensures the device can sustain loads between about 10 N·m and 60 N·m without slippage, enabling safe operation during dynamic movement or prolonged tasks. The integration of hardened or surface-treated materials, such as anodized aluminum, PTFE-lined polymer composites, and nitrided steel, ensures long service life under cyclic loading conditions exceeding 50,000 operational adjustments.

Another advantage is the device's modular and scalable construction, allowing components such as the arms 108, 508A, 508B, and load-distribution support elements 700 to be replaced, resized, or configured for single-arm or dual-arm use. The telescoping and overlapping arm assemblies (FIGS. 5A-5B) provide extension ranges between 100 mm and 250 mm, allowing the system to accommodate users of various sizes or to support differing activities—such as reading, operating tools, or supporting small electronic devices. This modularity also simplifies manufacturing and maintenance, as each component can be fabricated independently and assembled using standard fasteners. The elongate slot member 114A (FIG. 6) contributes to linear traversability and continuous spatial positioning of connected components. The combination of sliding channels, friction-locking knobs, and rotational couplings allows smooth translational adjustment with locking torques between about 150 N and 400 N, maintaining positional stability under load. This feature enables the exoskeleton support device 100 to be used effectively in both static and dynamic work environments.

The load-distribution support element 700 (FIG. 7) provides biomechanical benefits by redistributing the gravitational load of the user's forearm or mounted object through the mechanical framework rather than through the user's musculature. The concave upper surface, with curvature radii between 40 mm and 120 mm, cradles the limb and promotes even pressure distribution, reducing localized stress on the elbow and wrist. Multi-material construction, comprising rigid bases with viscoelastic or foam interfaces, ensures both load-bearing strength and user comfort, supporting continuous wear for durations exceeding 2-3 hours without discomfort.

In some embodiments, the exoskeleton support device may include one or more interchangeable attachments configured to replace one or more of the standard load-distribution pads. These interchangeable components may provide enhanced functional versatility, allowing the system to support a wider range of daily, occupational, or rehabilitative activities. For example, a removable or screw-on table-like platform may be secured to the distal end of an elongate slot member in place of the standard ergonomic pad. The platform may include a generally planar surface having a width of about 100 mm to about 400 mm, a length of about 150 mm to about 500 mm, and a thickness of about 3 mm to about 20 mm. In some embodiments the platform may support loads of about 0.5 kilograms to about 3 kilograms. The mounting interface may include a threaded stem measuring about 8 mm to about 20 mm in diameter, a quick-release coupling, or a friction-locking latch. The table surface may also include a raised perimeter edge between about 1 mm and about 10 mm high, or a textured surface friction coefficient of about 0.3 to about 0.8, to prevent objects such as pens, notebooks, or small tools from slipping.

In further embodiments, an enlarged pad or platform may be provided to support electronic devices. Such a platform may include a support surface area between about 150 square centimeters and about 600 square centimeters and may be sized to hold large smartphones measuring about 60 mm to about 100 mm wide or tablets measuring about 120 mm to about 300 mm wide. The platform may incorporate adjustable holding mechanisms including elastic retaining straps, pivoting clamps with opening ranges of about 5 mm to about 40 mm, or magnetic retention plates measuring about 20 mm to about 60 mm in diameter. The platform may also allow angular adjustment between about 0 degrees and about 120 degrees and rotational movement about a vertical axis between about 180 degrees and about 360 degrees.

The interchangeable attachments may be formed from lightweight materials such as polycarbonate, ABS polymer, aluminum alloy, or carbon-fiber composite. The weight of a given attachment may range from about 50 grams to about 500 grams depending on size and material. In some embodiments the attachment stem may provide rotational adjustment resistance between about 0.5 newton meters and about 5 newton meters to maintain stable orientation of the platform under load. Additional accessory options may include writing surfaces with a clipboard, a small instrument tray, a cup holder with internal diameters of about 50 mm to about 90 mm, and/or one or more auxiliary lighting mounts configured to receive LED modules. All accessory components may be configured to mate with the same connector geometry used by the standard load-distribution pads, enabling rapid user customization while maintaining proper load transfer and mechanical alignment throughout the exoskeleton framework.

The inclusion of spacer elements 800 (FIG. 8) between adjacent arm flex adjusters ensures consistent parallel alignment, smooth rotational movement, and minimized mechanical friction between stacked or interconnected arms. The spacer's polymeric construction eliminates the need for external lubrication, providing a low-maintenance and noise-dampened operation during repeated adjustments. The adjustable chest strap 900 (FIG. 9) and optional neck strap enable secure torso anchoring of the device without restricting user mobility. The strap design distributes mechanical loads across a broad surface area of the chest and shoulders, preventing point pressure and ensuring consistent posture support. The combination of multiple adjustment apertures, ratcheting or buckle systems, and optional elastic segments allows individualized fitment for users with chest circumferences between approximately 750 mm and 1200 mm.

In practical operation, as depicted in FIG. 10, the exoskeleton support device 100 permits hands-free interaction with electronic devices or tools positioned at adjustable distances and angles relative to the user's body. The device's structure maintains neutral upper-limb alignment, reducing deltoid and trapezius muscle activation by up to 40-60% compared to unsupported conditions, as measured in laboratory evaluations of upper-body assistive frameworks. This ergonomic load redistribution mitigates fatigue, improves posture, and promotes comfort during extended use in professional, rehabilitative, or daily living scenarios.

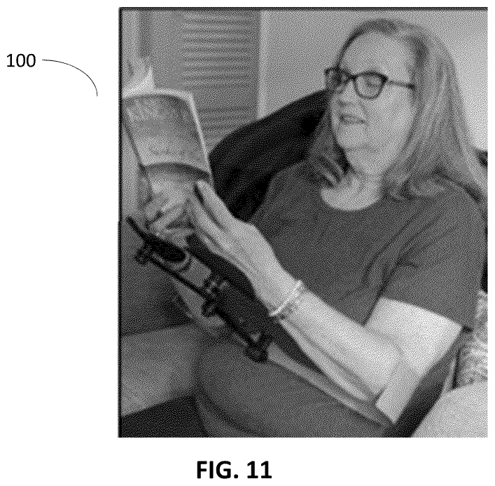

Referring to FIG. 11, the exoskeleton support device 100 is shown worn by a user in a resting or activity posture, demonstrating its dual-arm configuration during operation. In this embodiment, the system includes a pair of elongate slot members 114A and 114B, each extending outward from the support plate 102 and attached to corresponding load-distribution support elements 700 (see FIG. 7). These elements are positioned beneath the user's forearms and hands, thereby supporting their weight and reducing muscular strain during tasks such as reading, writing, typing, or device interaction. The dual elongate slot member configuration allows for independent or synchronized adjustment of each arm assembly. Each slot member 114A, 114B includes a longitudinal channel and a sliding fastener mechanism enabling linear adjustment of the forearm supports relative to the torso-mounted frame. The travel range along each slot may extend between 80 mm and 180 mm, preferably between 100 mm and 140 mm, providing user-specific reach customization. The locking mechanism may comprise a manual knob, detent lever, or threaded fastener, capable of securing the assembly under forces between about 150 N and 400 N.

Each slot member 114A, 114B terminates at a load-distribution support element 700 configured with a concave surface that cradles the underside of the user's forearm or wrist. The concave geometry, with a radius of curvature between about 60 mm and 100 mm, allows the user's forearm to rest in a neutral alignment, neither pronated nor supinated, thereby maintaining comfort and circulation during prolonged use. The paired support elements may also include soft overmolded or padded contact surfaces fabricated from memory foam, silicone gel, or thermoplastic elastomer (TPE), typically between 3 mm and 8 mm thick, to minimize contact pressure.

The dual elongate slot members 114A and 114B are connected at their proximal ends to the support plate 102 via indexed pivot joint couplings 110 (see FIG. 3), enabling each support assembly to rotate independently within an angular range of approximately 30° to 90° relative to the central axis of the frame. This rotational freedom allows the user to reposition each arm support laterally or vertically to achieve a comfortable and balanced configuration. In the embodiment shown, the exoskeleton support device 100 is worn without visibly showing the chest strap 900 (see FIG. 9) for clarity. In actual use, the chest strap would be fitted around the user's upper torso to secure the system in place. The user's forearms are positioned atop the load-distribution support elements 700, while the elongate slot members 114A and 114B transmit load through the pivot joints 110 and support plate 102, ultimately redistributing mechanical forces into the torso harness. This arrangement significantly reduces the static muscular effort required from the deltoid and biceps muscles, particularly during tasks requiring steady arm positioning.

The dual-arm system further allows symmetrical weight distribution, ensuring that neither side of the user's upper body bears excessive load. This design improves postural alignment, reducing shoulder elevation asymmetry and preventing fatigue commonly associated with unsupported activities such as holding a book, tablet, or kitchen utensil for extended periods. The supports can be angled upward, downward, or laterally outward to match different body geometries or activity contexts. The exoskeleton support device 100 may be constructed from lightweight aluminum alloy, carbon-fiber-reinforced polymer, or glass-filled nylon, maintaining total device mass below 1.5 kg while preserving structural stiffness sufficient to support static loads of up to 3.0 kg across both arms. The use of low-friction polymer bushings or washers within each pivot and slot interface allows for smooth adjustment with minimal mechanical noise.

In use, as shown in FIG. 11, the dual-slot configuration enables the user to maintain both hands in a relaxed, elevated position while reading a book or performing a fine-motor task. The hands-free support allows the user to sustain this posture for extended durations, typically up to 60 minutes or more, without perceivable fatigue. The modular design also allows one or both supports to be detached or repositioned depending on the activity, offering a versatile assistive function for daily living or rehabilitative applications. Accordingly, the embodiment illustrated in FIG. 11 demonstrates how the exoskeleton support device 100, through its dual elongate slot member arrangement, provides balanced ergonomic support, customizable alignment, and load-relieving functionality adaptable to a wide range of user needs and physical activities.

### EXAMPLES

Clause 1. An exoskeleton support device, comprising: a support plate with a proximal end located opposite a distal end, the support plate comprising: an opening located at the distal end of the support plate; and a first ratcheted coupling portion located at the proximal end of the support plate; an indexed pivot joint coupling having a first end located opposite a second end, the indexed pivot coupling comprising: a second ratcheted coupling portion located at the first end of the indexed pivot joint coupling, wherein the second ratcheted coupling portion of the indexed pivot joint coupling is configured to interlock at one or more discrete angular positions with the first ratcheted coupling portion of the support plate; and one or more arms each having a concave support member, the one or more arms being connected to the second end of the indexed pivot joint coupling; an elongate slot member having a first end located opposite a second end, the first end of the elongate slot member being connected to the support plate; and a load-distribution support element connected to the second end of the elongate slot member.

Clause 2. The exoskeleton support device of clause 1, wherein the first ratcheted coupling portion of the support plate having a plurality of radial teeth.

Clause 3. The exoskeleton support device of clause 1, wherein the second ratcheted coupling portion of the indexed pivot joint coupling having a plurality of radial teeth.

Clause 4. The exoskeleton support device of clause 1, wherein the second end of the indexed pivot joint coupling comprises an opening configured to receive a threaded fastener.

Clause 5. The exoskeleton support device of clause 1, further comprising: an opening located at the second end of the indexed pivot joint coupling configured to receive at least a portion of a threaded fastener for connecting at least one arm of the one or more arms to the indexed pivot joint coupling.

Clause 6. The exoskeleton support device of clause 1, further comprising: a first arm of the one of more arms is disposed over a surface of a second arm and is configured to expand and retract to accommodate differing lengths.

Clause 7. The exoskeleton support device of clause 1, wherein the elongate slot member is configured to provide linear adjustability between a pivot joint and a fixed mounting point.

Clause 8. The exoskeleton support device of clause 1, wherein the load-distribution support element further comprises a connector stem extending from a lower portion of the load-distribution support element and configured to couple the load-distribution support element to the second end of the elongate slot member to permit rotational or angular adjustment of the load-distribution support element relative to the elongate slot member.

Clause 9. The exoskeleton support device of clause 1, wherein the load-distribution support element comprises a concave upper surface configured to receive and cradle a hand of a user.

Clause 10. An exoskeleton support system, comprising: a frame configured to be worn by a user; a support plate having a proximal end located opposite a distal end, the support plate comprising a ratcheted coupling portion positioned at the proximal end; an indexed pivot joint coupling having a first end located opposite a second end, the first end of the indexed pivot joint coupling comprising a complementary ratcheted coupling portion configured to interlock with the ratcheted coupling portion of the support plate at one or more discrete angular positions; an arm assembly connected to the second end of the indexed pivot joint coupling, the arm assembly comprising a concave support member configured to support an arm region of the user; an elongate slot member having a first end located opposite a second end, the first end of the elongate slot member being connected to the support plate; and a load-distribution support element connected to the second end of the elongate slot member, the load-distribution support element comprising a padded upper surface configured to distribute load from the arm region of the user and a connector stem configured to couple the load-distribution support element to the elongate slot member.

Clause 11. The exoskeleton support system of clause 10, wherein the padded upper surface of the load-distribution support element comprises a resilient material selected from the group consisting of foam, gel, and elastomeric compounds.

Clause 12. The exoskeleton support system of clause 10, wherein the connector stem comprises a ball-and-socket joint configured to permit multi-directional angular adjustment of the load-distribution support element relative to the elongate slot member.

Clause 13. The exoskeleton support system of clause 12, wherein the ball-and-socket joint further comprises a locking mechanism configured to secure the load-distribution support element at a desired angular orientation.

Clause 14. The exoskeleton support system of clause 10, wherein the elongate slot member comprises graduated position markings configured to indicate relative adjustment positions of the load-distribution support element along a linear path.

Clause 15. The exoskeleton support system of clause 10, wherein the concave support member of the arm assembly comprises a replaceable ergonomic pad detachably coupled to an upper surface of the concave support member.

Clause 16. The exoskeleton support system of clause 10, wherein the arm assembly further comprises an outer tubular member and an inner telescoping member configured to adjust a length of the arm assembly.

Clause 17. The exoskeleton support system of clause 16, wherein the inner telescoping member comprises a detent locking pin configured to secure the arm assembly at one or more discrete extension positions.

Clause 18. The exoskeleton support system of clause 10, wherein the support plate and the indexed pivot joint coupling are formed of lightweight composite or polymeric material configured to reduce overall system weight.

Clause 19. The exoskeleton support system of clause 10, wherein the ratcheted coupling portion of the support plate and the complementary ratcheted coupling portion of the indexed pivot joint coupling are configured to lock at angular increments between about 5 degrees and about 15 degrees.

Clause 20. The exoskeleton support system of clause 10, wherein the frame, the arm assembly, and the elongate slot member are cooperatively configured to transfer and distribute mechanical load from the load-distribution support element through the frame to a torso region of the user, thereby reducing localized strain on an elbow, forearm, or wrist of the user.

Clause 21. An exoskeleton support system, comprising: a frame configured to be worn by a user; a support plate having a proximal end located opposite a distal end, the support plate comprising a ratcheted coupling portion positioned at the proximal end; an elongate slot member having a first end located opposite a second end, the first end of the elongate slot member being connected to the support plate; and a load-distribution support element connected to the second end of the elongate slot member; and a connector stem configured to couple the load-distribution support element to the elongate slot member.

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. An exoskeleton support device, comprising:
a support plate with a proximal end located opposite a distal end, the support plate comprising:
an opening located at the distal end of the support plate; and
a first ratcheted coupling portion located at the proximal end of the support plate;
an indexed pivot joint coupling having a first end located opposite a second end, the indexed pivot coupling comprising:
a second ratcheted coupling portion located at the first end of the indexed pivot joint coupling,
wherein the second ratcheted coupling portion of the indexed pivot joint coupling is configured to interlock at one or more discrete angular positions with the first ratcheted coupling portion of the support plate; and
one or more arms each having a concave support member, the one or more arms being connected to the second end of the indexed pivot joint coupling;
an elongate slot member having a first end located opposite a second end, the first end of the elongate slot member being connected to the support plate; and
a load-distribution support element connected to the second end of the elongate slot member.

2. The exoskeleton support device of claim 1, wherein the first ratcheted coupling portion of the support plate having a plurality of radial teeth.

3. The exoskeleton support device of claim 1, wherein the second ratcheted coupling portion of the indexed pivot joint coupling having a plurality of radial teeth.

4. The exoskeleton support device of claim 1, wherein the second end of the indexed pivot joint coupling comprises an opening configured to receive a threaded fastener.

5. The exoskeleton support device of claim 1, further comprising:
an opening located at the second end of the indexed pivot joint coupling configured to receive at least a portion of a threaded fastener for connecting at least one arm of the one or more arms to the indexed pivot joint coupling.

6. The exoskeleton support device of claim 1, further comprising:
a first arm of the one of more arms is disposed over a surface of a second arm and is configured to expand and retract to accommodate differing lengths.

7. The exoskeleton support device of claim 1, wherein the elongate slot member is configured to provide linear adjustability between a pivot joint and a fixed mounting point.

8. The exoskeleton support device of claim 1, wherein the load-distribution support element further comprises a connector stem extending from a lower portion of the load-distribution support element and configured to couple the load-distribution support element to the second end of the elongate slot member to permit rotational or angular adjustment of the load-distribution support element relative to the elongate slot member.

9. The exoskeleton support device of claim 1, wherein the load-distribution support element comprises a concave upper surface configured to receive and cradle a hand of a user.

10. An exoskeleton support system, comprising:
a frame configured to be worn by a user;
a support plate having a proximal end located opposite a distal end, the support plate comprising a ratcheted coupling portion positioned at the proximal end;
an indexed pivot joint coupling having a first end located opposite a second end, the first end of the indexed pivot joint coupling comprising a complementary ratcheted coupling portion configured to interlock with the ratcheted coupling portion of the support plate at one or more discrete angular positions;
an arm assembly connected to the second end of the indexed pivot joint coupling, the arm assembly comprising a concave support member configured to support an arm region of the user;
an elongate slot member having a first end located opposite a second end, the first end of the elongate slot member being connected to the support plate; and
a load-distribution support element connected to the second end of the elongate slot member; and
a connector stem configured to couple the load-distribution support element to the elongate slot member.

11. The exoskeleton support system of claim 10, further comprising:
a padded upper surface of the load-distribution support element comprises a resilient material selected from the group consisting of foam, gel, and elastomeric compounds.

12. The exoskeleton support system of claim 10, wherein the connector stem comprises a ball-and-socket joint configured to permit multi-directional angular adjustment of the load-distribution support element relative to the elongate slot member.

13. The exoskeleton support system of claim 12, wherein the ball-and-socket joint further comprises a locking mechanism configured to secure the load-distribution support element at a desired angular orientation.

14. The exoskeleton support system of claim 10, wherein the elongate slot member comprises graduated position markings configured to indicate relative adjustment positions of the load-distribution support element along a linear path.

15. The exoskeleton support system of claim 10, wherein the concave support member of the arm assembly comprises a replaceable ergonomic pad detachably coupled to an upper surface of the concave support member.

16. The exoskeleton support system of claim 10, wherein the arm assembly further comprises an outer tubular member and an inner telescoping member configured to adjust a length of the arm assembly.

17. The exoskeleton support system of claim 16, wherein the inner telescoping member comprises a detent locking pin configured to secure the arm assembly at one or more discrete extension positions.

18. The exoskeleton support system of claim 10, wherein the support plate and the indexed pivot joint coupling are formed of lightweight composite or polymeric material configured to reduce overall system weight.

19. The exoskeleton support system of claim 10, wherein the ratcheted coupling portion of the support plate and the complementary ratcheted coupling portion of the indexed pivot joint coupling are configured to lock at angular increments between about 5 degrees and about 15 degrees.

20. An exoskeleton support system, comprising:
a frame configured to be worn by a user;
a support plate having a proximal end located opposite a distal end, the support plate comprising a ratcheted coupling portion positioned at the proximal end;
an elongate slot member having a first end located opposite a second end, the first end of the elongate slot member being connected to the support plate; and
a load-distribution support element connected to the second end of the elongate slot member; and
a connector stem configured to couple the load-distribution support element to the elongate slot member.
